(19) United States
(12) Patent Application Publication
(10) Pub. No.: US 2019/0284176 A1
(43) Pub. Date: Sep. 19, 2019

(54) COMPOSITIONS AND METHODS FOR TREATING CARDIOMYOPATHY

(71) Applicant: CASE WESTERN RESERVE UINVERSITY, Cleveland, OH (US)

(72) Inventors: Mukesh K. Jain, Solon, OH (US); Xudong Liao, Solon, OH (US)

(21) Appl. No.: 16/358,259

(22) Filed: Mar. 19, 2019

Related U.S. Application Data
(60) Provisional application No. 62/644,792, filed on Mar. 19, 2018.

(51) Int. Cl. C07D 405/14; A61K 47/59; C07K 14/715

(52) U.S. Cl. CPC C07D 405/14; C07K 14/715; A61K 47/59

(57) ABSTRACT

A method of treating a cardiomyopathy in a subject in need thereof includes administering to the subject a therapeutically amount of a CCR2 inhibitor.

### RELATED APPLICATION

This application claims priority from U.S. Provisional Application No. 62/644,792, filed Mar. 19, 2018, the subject matter of which is incorporated herein by reference in its entirety.

### BACKGROUND

Cardiomyopathy is a disease of the heart muscle that decreases the heart's ability to pump blood and meet the body's energetic demands. Cardiomyopathy can be broadly classified as ischemic cardiomyopathy (ICM) or nonischemic cardiomyopathy (NICM). In ICM, interruption of coronary artery blood flow, as seen in myocardial infarction (MI), is the primary cause of cardiac dysfunction. In NICM, cardiac muscle function can be compromised by either intrinsic gene mutation (hypertrophic cardiomyopathy) or extrinsic stress, such as pressure overload secondary to chronic hypertension or valve diseases [pressure overload hypertrophy (POH)]. With respect to NICM, accumulating clinical evidence suggests that small vessel dysfunction is a strong independent predictor of clinical deterioration and death in patients. Further, experimental studies suggest that inadequate myocardial angiogenesis is a determinant of the transition from compensatory hypertrophy to heart failure.

Macrophages are found in virtually every tissue and are critical for homeostasis and stress-induced responses. Macrophages broadly consist of two classes: resident macrophages and bloodborne infiltrating macrophages. Resident macrophages originate from yolk sac-derived erythromyeloid progenitors (EMPs), reside in tissue, perform homeostatic functions, and self-maintain locally. In general, infiltrating macrophages arise from circulating classic Ly6Chi (inflammatory) monocytes and are recruited after inciting pathology through the CCL2-CCR2 chemotaxis pathway. Nonclassic Ly6Clo (patrolling) monocytes also patrol the luminal side of the endothelium and extravasate in response to both septic and aseptic tissue injury, playing a protective or antiinflammatory role during tissue injury. The adult heart contains two major subsets of Ly6Clo/CCR2− resident macrophages that express different levels of MHC-II, which are EMP-derived resident macrophages self-maintained through local proliferation at steady state. In addition, there are two minor subsets of Ly6Chi macrophages (<2%) that differ in CCR2 levels. These Ly6Chi subsets are derived from hematopoietic stem cells, are maintained through monocyte infiltration, and help to resolve tissue injury.

### SUMMARY

Embodiments described herein relate to compositions and methods for use in the prevention and treatment of cardiomyopathy and/or heart failure and, particularly, nonischemic cardiomyopathy and/or heart failure. It was found that cardiac resident macrophage proliferation and angiogenic activity can be essential for cardiac adaptation and function in an initial period of heart failure. Furthermore, cardiac resident macrophages are essential to maintain cardiac function even in the late phase of heart failure. KLF4 was identified as a regulator of resident macrophage proliferation and angiogenic function and its deficiency or loss resulted in a marked reduction in local proliferation of cardiac resident macrophages and the total number of cardiac macrophages. In the late phase of heart failure, infiltrating monocytes/macrophages promote decompensation and blockade of this ingress was found to be ameliorative.

Accordingly, in some embodiments a method of treating a cardiomyopathy and/or heart failure in a subject in need thereof includes administering to the subject a therapeutically effective amount of a CCR2 inhibitor. The CCR2 inhibitor can be administered at an amount effective to preserve cardiac function.

In some embodiments, the cardiomyopathy is nonischemic cardiomyopathy. The CCR2 inhibitor can be administered to the subject after the occurrence of the cardiomyopathy or cardiac hypertrophy to inhibit infiltrations of blood-borne macrophages into myocardium of the subject. The blood-borne macrophages can include, for example, Ky6C^{hi}, CX3CR1⁺, CCR2⁺ macrophages. The CCR2 inhibitor can also be administered to the subject during late-phase pressure overload hypertrophy.

In some embodiments, the CCR2 inhibitor can be selected from the group consisting of: (i) a direct CCR2 antagonist; (ii) an inverse CCR2 agonist; (iii) a negative allosteric CCR2 modulator; (iv) an indirect CCR2 antagonist; (v) an indirect inverse CCR2 agonist; and (vi) an indirect negative allosteric CCR2 modulator. For example, the CCR2 inhibitor can be a CCR2 antagonist selected from the group consisting of RS504393, RS102895, MLN-1202, INCB8696, MK-0812, CCX140, PF-4136309, and BMS-741672.

In other embodiments, an agent can be administered to the subject that promotes expression Kruppel-like factor 4 (KLF4) in myocardium of the subject. The agent can be administered to the subject at an amount effective promote to cardiac resident macrophage proliferation and angiogenic activities. The agent can include a vector encoding KLF4.

### BRIEF DESCRIPTION OF THE DRAWINGS

FIGS. 1(A-I) illustrate pressure overload induces local proliferation of cardiac resident macrophages at early phase and infiltration of monocytes at late phase. (A) Gating strategy for flow cytometry. (B) Dynamics of cardiac macrophages following TAC and its correlation with cardiac function. The dashed line indicates baseline. Cardiac macrophages were gated as live (live/dead stain-negative) CD45+CD11b+Ly6G−F4/80+CD64+ cells and are shown as a percentage vs. total heart cells. Cardiac function was expressed as left ventricular fractional shortening. (C) Immunostaining for Mac-3 to reveal macrophages in the myocardium, particularly in the perivascular region. Brown (DAB-positive) cells are macrophages. Arrowheads indicate vessels. (Scale bars, 100 μm) (D) LV dysfunction following TAC. (E) Strategy to generate CD45.1-CD45.2 chimeric mice. Recipient CD45.2 mice were irritated with their head-chest shielded by lead block and transplanted via tail vein injection with bone marrow cells from CD45.1 donor mice. The resulting chimera mice carry both CD45.2 and CD45.1 cells in the blood but only have CD45.2 cardiac resident macrophages. (F) Blood (50 μL) was drawn from the tail vein at 8 wk posttransplantation and analyzed by FACS for CD45.1+ myeloid cells (CD45+CD11b+). (G) FACS analysis for CD45.1+ cardiac macrophages before and after TAC. (H) Corrected monocyte infiltration rate considering that only 50% of monocytes were CD45.1+ cells. (I) FACS analysis for Ki-67 expression in cardiac macrophages. (B and D) P values were calculated from one-way ANOVA. (F-I) *P<0.05 by t test after Bonferroni correction (n=5-8 for all data points). NS, not significant.

FIGS. 2(A-F) illustrate local proliferation of cardiac resident macrophages and infiltration of blood-borne Ly6C+ monocytes. (A and B) TAC induced local proliferation of cardiac resident macrophages as assessed by BrdU pulse-labeling assay. DNA was stained with Hoechst 33342 for cycle analysis, and the S-phase was clearly visible in TAC 3-d samples. The time course curve of total cardiac macrophages (dotted red line) was overlaid to show the phase difference. (C and D) Monocytes (CD115+/CD1 lb+) in blood, spleen, and bone marrow before and after TAC. (E and F) Classic Ly6C+ monocytes and nonclassic Ly6C− monocytes before and after TAC. In B, P values were calculated from one-way ANOVA for BrdU data. In D and F, *P<0.05 by t test (n=5 for all data points). NS, not significant.

FIGS. 3(A-I) illustrate cardiac resident macrophages are requisite for cardiac adaptation to POH. (A) Blocking monocyte infiltration by RS had no effect on POH. (B) Left ventricular function following clodronate-mediated macrophage depletion and TAC (n=8-10 in each group). The P value was calculated from two-way ANOVA. The blue arrow indicates the time of TAC, and red arrows indicate the time of liposome injections. (C) Postoperation survival rates. P values were calculated from log-rank between TAC+CL and TAC+Veh groups. (D-H) Gene expression in the heart after 4-d TAC±CL (n=5). *P<0.05 by t test with Bonferroni correction. (I) Cell death detected by TUNEL staining in the myocardium after 4-d TAC±CL. Red arrowheads indicate nuclei of dead cells. *P<0.05 by t test (n=5). NS, not significant. (Scale bars, 200 μm.)

FIGS. 4(A-H) illustrate myeloid KLF4 regulates cardiac macrophage proliferation in POH. (A) RNA-seq studies with primary peritoneal macrophages from Cre and K4-cKO mice. A total of 283 genes were differentially regulated between the Cre and K4-cKO groups, and gene ontology (GO) term enrichment analysis revealed nine pathways that were functionally enriched [false discovery rate (FDR)<0.05]. Arrows indicate pathways of interest. (B) KLF4-deficient peritoneal macrophages exhibited an impaired proliferative response to CSF1/2 stimulation (48 h). *P<0.05. (C) KLF4-deficiency impaired TAC-induced cardiac macrophage proliferation (n=10). *P<0.05. (D) Myeloid KLF4 deficiency impaired cardiac function after TAC, resulting in dilated cardiomyopathy at 2 wk post-TAC (n=8-10). *P<0.05. EDV, end-diastolic volume; ESV, end-systolic volume; LVEF, left ventricular ejection fraction. (E) Myeloid KLF4 deficiency accelerated TACinduced cardiac hypertrophy. Heart weight (HW; milligrams) and lung weight (LW; milligrams) at 2 wk post-TAC were normalized to body weight (grams) (n=6 in each group). *P<0.05 by t test with Bonferroni correction. (F) TAC induced more fibrosis, cell death, and mitochondrial damage in K4-cKO hearts. Samples were from 1-wk TAC and sham mice. (Black scale bar, 100 μm; white scale bar, 2 μm) (G) TAC induced higher plasma cardiac troponin I levels in the K4-cKO group (n=5). *P<0.05 with Bonferroni correction. (H) Impaired angiogenesis in K4-cKO hearts after TAC as revealed by myocardial capillary density using CD31 immunostaining (n=5). *P<0.05. (Scale bars, 100 μm.).

FIGS. 5(A-I) illustrate blockade-infiltrating macrophages preserved cardiac function in late-phase POH. (A) Classic Ly6C+CCR2+ monocytes increased in blood after 4 wk of TAC. A representative FACS plot from five samples is shown. (B) Reduced monocyte numbers in CCR2-KO mice after 4 wk of TAC (n=5). *P<0.05 by t test. (C) Reduced cardiac macrophage numbers in CCR2-KO hearts after 4 wk of TAC (n=5). *P<0.05 by t test. (D) Left ventricular function assessed by echocardiography. The P value is shown as P (genotype*time) calculated from two-way ANOVA. (E) Heart weight and lung weight. (F) Expression of hypertrophy marker genes. (G) Cardiomyocyte cross-section area. Cell membranes were outlined by wheat germ agglutinin (WGA) staining. (H) Fibrosis assessed by PicoSirus Red staining, with collagen stained in red. Perivascular and intramuscular areas are shown. Fibrosis was quantified as the percentage of the fibrotic area in total by ImageJ (NIH) software. (I) Myocardial capillary density assessed by CD31 staining. All tissue samples were assessed at 8 wk post-TAC (n=5-8). *P<0.05. NS, not significant. (Scale bars, 200 μm.)

FIG. 6 illustrates dynamic changes and distinct roles of cardiac macrophage subsets in POH. Our studies have demonstrated a two-phase response of cardiac macrophages to pressure overload. (i) During the early phase of POH, there is robust local proliferation of resident macrophages that is crucial for cardiac compensation to pressure overload. (ii) During the late phase of POH, the infiltration of inflammatory monocytes increases, which promotes the transition to cardiac decompensation. Mechanistically, transcription factor KLF4 is required for resident macrophage proliferation and the infiltrating monocytes are Ly6ChiCX3CR1+CCR2+ classic monocytes. Finally, this model implies that these two subsets of cardiac macrophages can be targeted for therapeutic gain, either by blocking monocytes (i.e., RS administration) or by promoting resident macrophages (i.e., prolonged proliferation), or by simultaneously targeting both subsets for a healthy balance.

FIGS. 7(A-D) illustrate TAC did not induce significant monocyte infiltration in early-phase POH. (A) FACS analysis of cardiac macrophages. MHC-II and CCR2 were plotted to show resident macrophages. (B) The CCR2− resident macrophages remained as >90% after TAC, indicating no significant infiltration of CCR2+ monocytes at 1-week post-TAC. RS-504393 (RS) dissolved in PBS+5% DMSO was administrated by oral gavage in one set of animals to block CCR2-mediated chemotaxis. PBS+5% DMSO was used as vehicle (Veh) control. (C & D) Majority of cardiac macrophages are CD64+Ly6C− before and after 1-week TAC. n=5.

FIG. 8 illustrates two-hour BrdU pulse-labeling. Blood cells were not labelled by BrdU within two hours of BrdU injection. BrdU incorporation into DNA was detected by APC-conjugated anti-BrdU antibody using FACS.

FIGS. 9(A-B) illustrate clodronate liposome (CL)-mediated macrophage depletion. (A) Organs from mice receiving 3 injections of control PBS-containing liposomes (Veh) and CL containing liposomes (CL). Surgery was performed 1-day after first injection. Organ weight (in mg) was normalized to body weight (BW, in gram). HW: heart weight. LW: lung weight. SW: spleen weight. n=5, *p<0.05. (B) Histology of hearts after 4-day TAC and 3 injections of liposomes (Veh or CL). Cardiac hypertrophy was assessed by WGA staining. Fibrosis was assessed by PicoSirus Red staining. Collagen was stained in red. Myocardial capillary density was assessed by CD31 staining. Scale bar indicates 200 μm. All tissue samples were assessed at 4-day post-TAC (and after 3 injections).

FIGS. 10(A-B) illustrate TAC induced non-apoptotic death of cardiomyocytes in macrophage-depleted (CL) heart. (A) Fluorescent TUNEL staining coupled with anti-cardiac actin immunostaining. TUNEL signal (dead nuclei). Cardiac actin. DAPI. Scale bar indicates 20 μm. (B) Immunostaining for cleaved Caspase 3 in the myocardium. (DAB-positive) cells were highlighted with red arrow head. Scale bar indicates 100 μm. All tissue samples were assessed at 4-day post-TAC (and after 3 injections).

FIGS. 11(A-F) illustrate macrophage depletion (CL) after 2 weeks of TAC induced quick transition to heart failure and death. (A) Post-TAC survival rates. Blue arrow indicates time of TAC and red arrows indicate time of liposome injections. P value was calculated from Log-rank test. n=10 in each group. Veh: PBS liposomes; CL: Clodronate liposomes. (B) Echocardiography. *p<0.05 by t test. n=5. (C) Cardiomyocyte hypertrophy (WGA staining) and fibrosis (Sirius Red staining). Scale bar indicates 100 μm. (D) Cell death in the myocardium assessed by TUNEL staining. Scale bar indicates 100 μm. (E) Cardiac actin and TUNEL double staining confirmed cardiomyocyte death in TAC+CL heart. Scale bar indicates 25 μm. (F) Cleaved Caspase-3 staining showing very few apoptotic cells. Scale bar indicates 200 μm. Arrow heads indicate dead cells.

FIGS. 12(A-E) illustrate depletion of neutrophils exhibited little effect on POH. (A) LV function assessed by serial echocardiography. IgG: control antibody. 1A8: anti-Ly6G antibody, clone 1A8. Arrows: Antibody i.p. injection (red arrow) was started 1-day before TAC (blue arrow) and kept for 4 weeks during TAC. P value shown as P (treatment*time) calculated from Two-Way ANOVA. (B) heart weight (HW) and lung weight (LW) after 4-week TAC, normalized to body weight (BW). n=5. (C) Expression of hypertrophy marker genes (Nppa, Nppb and Acta1) in the heart after 4-week TAC. P<0.05 by t test with Bonferroni correction. n=5. (D) Fibrosis assessed by PicoSirus Red staining. Collagen was stained in red. Perivascular and intramuscular areas were shown. Scale bar indicates 100 μm (E) Neutrophil depletion was confirmed by FACS.

FIGS. 13(A-B) illustrate KLF4 deficiency impaired macrophage proliferation. (A) Peritoneal macrophages isolated from Cre and K4-cKO mice were treated with CSF1 or CSF2 (10 ng/ml) for 48 h before subjected to flow cytometry analysis for Ki-67 expression. DNA was stained by Hoeschst 33342. Gated cells were taken as Ki-67 positive populations. (B) BrdU labelling in cardiac macrophages after 1-week TAC. *p<0.05 by t test. n=5.

FIGS. 14(A-E) illustrate Myeloid KLF4 deficiency impaired angiogenesis. (A, B) Expression of angiogenic genes in the heart before and after TAC. (C) KLF4 expression in cardiac macrophages isolated from Cre and K4-cKO mice at 1-week post-surgery. (D) VEGFA expression in peritoneal and cardiac macrophages. (E) Expression of 84 fibrosis genes in cardiac macrophages isolated from Cre and K4-cKO mice at 1-week post-TAC. There was no gene that had both a fold change (FC=KO/Cre) over ±1.5 (dotted red lines) and p<0.05 (data point in red). For all data point, n=4 in each group, *p<0.05 between genotypes by t test with bonferroni correction.

FIGS. 15(A-D) illustrate deficiency of M2 macrophages did not affect POH. (A, B) Expression of M1 and M2 genes in the Lyz2-Cre and K4-cKO hearts in response to TAC. (C, D) The IL4-Stat6 signaling pathway is a key regulator of macrophage M2 polarization but mice with either hematopoietic deficiency of Stat6 (C) or myeloid-specific deficiency of IL4 receptor (IL4Ra-KO) (D) exhibited normal cardiac responses to TAC. n=5.

FIGS. 16(A-C) illustrate RS-504393 administration preserved cardiac function in established POH. (A) LV function assessed by echocardiography. P value shown as P (treatment*time) calculated from Two-Way ANOVA. RS-504393 (RS) administration started from day-14 post-TAC to bypass the early adaptation phase. Arrows indicate treatment starting time. (B) Heart weight (HW) and lung weight (LW) normalized to body weight (BW). (C) Expression of hypertrophy marker genes. All tissue samples were assessed at 6-week post-TAC. n=6, *p<0.05 by t test with Bonferroni correction.

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this application belongs.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

The term “or” as used herein should be understood to mean “and/or”, unless the context clearly indicates otherwise.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc.

The terms “prevent,” “preventing,” or “prevention” are art-recognized and include precluding, delaying, averting, obviating, forestalling; stopping, or hindering the onset, incidence, severity, or recurrence of a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

The term “pharmaceutical composition” refers to a formulation containing the disclosed compounds in a form suitable for administration to a subject. In a preferred embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., a formulation of the disclosed compound or salts thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, inhalational, and the like. Dosage forms for the topical or transdermal administration of a compound described herein includes powders, sprays, ointments, pastes, creams, lotions, gels, solutions, patches, nebulized compounds, and inhalants. In a preferred embodiment, the active compound is mixed under sterile conditions with a pharmaceutically acceptable carrier, and with any preservatives, buffers, or propellants that are required.

The term “flash dose” refers to compound formulations that are rapidly dispersing dosage forms.

The term “immediate release” is defined as a release of compound from a dosage form in a relatively brief period of time, generally up to about 60 minutes. The term “modified release” is defined to include delayed release, extended release, and pulsed release. The term “pulsed release” is defined as a series of releases of drug from a dosage form. The term “sustained release” or “extended release” is defined as continuous release of a compound from a dosage form over a prolonged period.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compounds described herein can also be prepared as prodrugs, for example pharmaceutically acceptable prodrugs. The terms “pro-drug” and “prodrug” are used interchangeably herein and refer to any compound, which releases an active parent drug in vivo. Since prodrugs are known to enhance numerous desirable qualities of pharmaceuticals (e.g., solubility, bioavailability, manufacturing, etc.) the compounds can be delivered in prodrug form. Thus, the compounds described herein are intended to cover prodrugs of the presently claimed compounds, methods of delivering the same and compositions containing the same. “Prodrugs” are intended to include any covalently bonded carriers that release an active parent drug in vivo when such prodrug is administered to a subject. Prodrugs are prepared by modifying functional groups present in the compound in such a way that the modifications are cleaved, either in routine manipulation or in vivo, to the parent compound. Prodrugs include compounds wherein a hydroxy, amino, sulfhydryl, carboxy, or carbonyl group is bonded to any group that may be cleaved in vivo to form a free hydroxyl, free amino, free sulfhydryl, free carboxy or free carbonyl group, respectively. Prodrugs can also include a precursor (forerunner) of a compound described herein that undergoes chemical conversion by metabolic processes before becoming an active or more active pharmacological agent or active compound described herein.

A “patient,” “subject,” or “host” to be treated by the subject method may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. In another aspect, the subject is a prematurely born mammal treated with prolonged supplemental oxygen. A patient refers to a subject afflicted with a disease or disorder.

The terms “prophylactic” or “therapeutic” treatment is art-recognized and includes administration to the host of one or more of the therapeutic compositions described herein. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation.

The term “cardiac hypertrophy” is used in its ordinary meaning as understood by the medical community and is often associated with increased risk of morbidity and mortality. It generally refers to the process in which adult cardiac myocytes respond to stress through hypertrophic growth. Such growth is characterized by cell size increases without cell division or proliferation, assembling of additional sarcomeres within the cell to maximize force generation, and an activation of a fetal cardiac gene program.

The term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. The terms “heart failure,” “manifestations of heart failure,” “symptoms of heart failure,” and the like are used broadly to encompass all of the sequelae associated with heart failure, such as shortness of breath, pitting edema, an enlarged tender liver, engorged neck veins, pulmonary rales and the like including laboratory findings associated with heart failure.

The term “nonischemic cardiomyopathy” refers to a disease of the myocardium associated with mechanical or electrical dysfunction exhibiting inappropriate ventricular hypertrophy or dilation. Nonischemic cardiomyopathy may be either primary (confined to the heart) or secondary to systemic conditions.

Embodiments described herein relate to compositions and methods for use in the prevention and treatment of cardiomyopathy and/or heart failure and, particularly, nonischemic cardiomyopathy and/or heart failure. It was found that cardiac resident macrophage proliferation and angiogenic activity can be essential for cardiac adaptation and function in an initial period of heart failure. Furthermore, cardiac resident macrophages are essential to maintain cardiac function even in the late phase of heart failure. KLF4 was identified as a regulator of resident macrophage proliferation and angiogenic function and its deficiency or loss resulted in a marked reduction in local proliferation of cardiac resident macrophages and the total number of cardiac macrophages. In the late phase of heart failure, infiltrating monocytes/macrophages promote decompensation and blockade of this ingress was found to be ameliorative.

Accordingly, in some embodiments a method of treating a cardiomyopathy and/or heart failure in a subject in need thereof includes administering to the subject a therapeutically effective amount of a CCR2 inhibitor. In some embodiments, the cardiomyopathy is nonischemic cardiomyopathy.

The CCR2 inhibitor can include any compound or agent (e.g., small molecules, ligands, proteins, enzymes, antibodies, nucleic acids, etc.) which inhibits or partially inhibits any one of the pathways associated with the CCR2 pathway, including compounds or agents which inhibit components of CCR2 pathway other than the chemokine receptor itself. For example, the inhibitor may inhibit or partially inhibit proteins that associate with chemokine receptors, or may inhibit compounds or pathway steps before and/or after the chemokine receptor itself.

In some embodiments, the CCR2 inhibitor can include any inhibitor which inhibits or partially inhibits any one of the chemokine receptor pathways associated with CCR2, which includes a direct CCR2 antagonist, inverse agonist or negative allosteric modulator, or an antagonist, inverse agonist or negative allosteric modulator which works indirectly through blocking of these pathways at different levels. For example, the CCR2 inhibitor can be a CCR2 antagonist selected from the group consisting of RS504393 (Sigma-Aldrich), RS102895 (Sigma-Aldrich), MLN-1202 (Millennium Pharmaceuticals), INCB8696 (Incyte Pharmaceuticals), MK-0812 (Merck), CCX140 (ChemoCentryx), PF-4136309 (Pfizer), and BMS-741672 (Bristol-Myers Squibb).

In some embodiments, a therapeutically effective amount of a CCR2 inhibitor can include an amount sufficient to preserve cardiac function. More specifically, a therapeutically effective amount can include an amount effective to inhibit infiltrations of blood-borne macrophages into myocardium of the subject. The CCR2 inhibitor can be administered to the subject after the occurrence or onset of the cardiomyopathy or cardiac hypertrophy to inhibit infiltrations of blood-borne macrophages into myocardium of the subject. For example, the CCR2 inhibitor can be administered at least one day, at least two days, at least 3 days, at least four days, at least five days, at least six days, at least one week, at least two weeks, at least three weeks, at least four weeks, at least five weeks, at least six weeks at least seven weeks, at least eight weeks or more after the occurrence or onset of the cardiomyopathy or cardiac hypertrophy to inhibit infiltrations of blood-borne macrophages into myocardium of the subject. The blood-borne macrophages can include, for example, Ky6C^{hi}, CX3CR1⁺, CCR2⁺ macrophages. The CCR2 inhibitor can also be administered to the subject during late-phase pressure overload hypertrophy to inhibit infiltrations of blood-borne macrophages into myocardium of the subject.

A therapeutically effective amount can vary based on a range of factors (e.g., route of administration, patient's age, patient's weight, severity of disorder, etc.) and determination thereof is well within the capability of those skilled in the art. For instance, a therapeutically effective amount of a CCR2 inhibitor can be estimated initially from cell culture assays. For example, a dose can be formulated in animal models to achieve a circulating concentration range that includes the IC₅₀ (the dose where 50% of the cells show the desired effects) as determined in cell culture. Such information can be used to more accurately determine useful doses in mammals (e.g., humans).

A therapeutically effective amount of a CCR2 inhibitor can also refer to that amount of the compound that results in amelioration of symptoms or a prolongation of survival in a patient. Toxicity and therapeutic efficacy of a CCR2 inhibitor can be determined by standard pharmaceutical procedures in cell cultures or experimental animals (e.g., for determining the LD50—the dose lethal to 50% of the population) and the ED50 (the dose therapeutically effective in 50% of the population). The dose ratio between toxic and therapeutic effects is the therapeutic index and it can be expressed as the ratio between LD50 and ED50. Compounds, which exhibit high therapeutic indices are preferred. The data obtained from these cell culture assays and animal studies can be used in formulating a range of dosages or amounts for use in mammals (e.g., humans). The dosage or amount of a CCR2 inhibitor preferably lies within a range of circulating concentrations that include the ED50 with little or no toxicity. The dosage or amount may vary within this range depending upon the dosage form employed and the route of administration utilized. The exact formulation, route of administration and dosage can be chosen by the individual physician in view of the patient's condition. Dosage amount and interval may be adjusted individually to provide plasma levels of the active moiety which are sufficient to maintain the desired effects.

In cases of local administration or selective uptake, the effective local concentration of the CCR2 inhibitor may not be related to plasma concentration.

The amount of CCR2 inhibitor containing composition administered can, of course be dependent upon several factors including the subject being treated, on the subject's weight, the severity of the affliction, the manner of administration and the judgment of the prescribing physician.

In certain embodiments, the methods can include the administration of a therapeutically effective amount of a CCR2 inhibitor, in which the amount of the CCR2 inhibitor agonist comprises an amount effective to preserve cardiac function and halt further cardiac cell hypertrophy. For instance, the rate at which hypertrophic cardiomyocyte growth can begin to reduce until no noticeable further growth is realized. In this regard, these embodiments can provide a means to effectively impede or stop the further progression or severity of cardiac hypertrophy and related pathologies.

In certain embodiments, the subject (e.g., human) being treated has been diagnosed as having cardiac hypertrophy. In other embodiments, however, the subject (e.g., human) being treated may not technically have cardiac hypertrophy but may be exhibiting symptoms similar to or associated with cardiac hypertrophy. In certain embodiments, the subject (e.g., human) being treated may be identified as being at risk of developing cardiac hypertrophy, cardiac hypertrophy associated cardiovascular disease and/or heart failure in view of diagnosis of conditions known to ultimately lead to development of cardiovascular disease. In such embodiments, the administration of a CCR2 inhibitor can beneficially facilitate or prevent further development of cardiac hypertrophy, cardiac hypertrophy associated cardiovascular disease and/or heart failure.

In some embodiments, the CCR2 inhibitor can provided in a pharmaceutical composition with a wide variety of pharmaceutically acceptable carriers or excipients. The particular carriers or excipients can be varied depending on various factors including route of administration, presence or absence of a carrier/targeting moiety, and desired delivery system (e.g., sustained release, timed-released, immediate release, selective release, etc.). For example, the composition can be made to suit the desired mode of administration. Pharmaceutically acceptable carriers can be determined, in part, by the particular composition being administered, as well as by the particular method used to administer the composition. Accordingly, there is a wide variety of suitable formulation recipes of pharmaceutical compositions containing one or more CCR2 inhibitors. For example, the pharmaceutical carrier may comprise a virus, a liposome (e.g., cationic lipids mixed with a CCR2 inhibitor to form liposomes), or a polymer (e.g., cationic polymers such as DEAE-dextran or polyethylenimine in which the CCR2 inhibitor complexes with the polycation and the complex is taken up by the cell via endocytosis).

The administration of a pharmaceutical composition that includes a CCR2 inhibitor may be carried out by known methods, wherein a desired molecule is introduced into a desired target cell in vitro or in vivo. In general, methods of administering small molecules, nucleic acids, enzymes and proteins are well known in the art. CCR2 inhibitor compositions in accordance with some embodiments can be administered by a number of routes including, but not limited to: oral, intravenous, intraperitoneal, intraarterial, intramuscular, transdermal, subcutaneous, topical, sublingual, or rectal means. Alternatively, the CCR2 inhibitor can be administered using a cellular vehicle, such as cells “loaded” with the CCR2 inhibitor ex vivo.

Administration of the compositions described herein may be accomplished by any acceptable method which allows a CCR2 inhibitor to reach its target. Any acceptable method known to one of ordinary skill in the art may be used to administer a composition to the subject. The administration may be localized (i.e., to a particular region, physiological system, tissue, organ, or cell type) or systemic, depending on the condition being treated. In certain embodiments, the targeted cells include blood-borne macrophages.

Injections can be, for example, intravenous, intradermal, subcutaneous, intramuscular, or intraperitoneal. In certain embodiments, the injections can be given at multiple locations if desired. In certain embodiments, the compositions can be delivered by implantation. Implantation can include inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. In certain embodiments, the compositions can be delivered orally or sublingually. In certain embodiments, the compositions can be delivered by inhalation. Inhalation can include administering the composition with an aerosol in an inhaler, either alone or attached to a carrier that can be absorbed. For systemic administration, it may be preferred that the composition is encapsulated in liposomes.

In some embodiments, the CCR2 inhibitor delivery systems can be provided in a manner which enables tissue-specific uptake of the CCR2 inhibitor. Techniques include using tissue or organ localizing devices, such asstents having drug delivery capability and configured as expansive devices or stent grafts.

In certain embodiments, a therapeutically effective amount of one or more CCR2 inhibitor can be delivered to a mammal via a nanoparticle-based drug delivery system. For instance, nanoparticles used as carriers for CCR2 inhibitor can provide the benefits of high stability, high carrier capacity, feasibility of incorporation of both hydrophilic and hydrophobic substances, and feasibility of variable routes of administration, including oral application and inhalation. In certain embodiments, the nanoparticles can also be designed to allow controlled (sustained) release of the CCR2 inhibitor from the matrix. The aforementioned properties of nanoparticles, according to certain embodiments of the present invention, can provide improvement of bioavailability and/or reduction of the dosing frequency. Nanoparticles for the purpose of CCR2 inhibitor delivery can be defined as submicron (<1 μm) colloidal particles. The colloidal particles can include monolithic nanoparticles (nanospheres) in which the CCR2 inhibitor is adsorbed, dissolved, or dispersed throughout the matrix and nanocapsules in which the CCR2 inhibitor is confined to an aqueous or oily core surrounded by a shell-like wall. Alternatively, the CCR2 inhibitor can be covalently attached to the surface or into the matrix. Nanoparticles, according to certain embodiments of the present invention, can be made from biocompatible and biodegradable materials such as polymers, either natural (e.g., gelatin, albumin) or synthetic (e.g., polylactides, polyalkylcyanoacrylates), or solid lipids. In the body of the mammal being treated, the CCR2 inhibitor loaded in nanoparticles can be released from the matrix by a variety of mechanisms including, for example, diffusion, swelling, erosion, degradation, or combinations thereof.

In certain embodiments, the compositions can be delivered using a bioerodible implant by way of diffusion or by degradation of a polymeric matrix. In certain embodiments, the administration of the compositions may be designed so as to result in sequential exposures to the CCR2 inhibitor over a certain time period, for example, hours, days, weeks, months or years. This may be accomplished, for example, by repeated administrations or by a sustained or controlled release delivery system in which a CCR2 inhibitor is delivered over a prolonged period without repeated administrations. Administration of the compositions using such a delivery system may be, for example, by oral dosage forms (e.g., tablet, capsule, etc.), bolus injections, transdermal patches or subcutaneous implants. Maintaining a substantially constant concentration of the CCR2 inhibitor agonist may be preferred in some cases.

Other delivery systems include, but are not limited to, time-release, delayed release, sustained release, or controlled release delivery systems (e.g., tablets, capsules, etc.). Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these.

Microcapsules of the foregoing polymers containing nucleic acids are described in, for example, U.S. Pat. No. 5,075,109. Other examples include nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include, but are not limited to, erosional systems in which a synthetic compound is contained in a formulation within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The compositions may be as, for example, microspheres, hydrogels, polymeric reservoirs, cholesterol matrices, or polymeric systems. In certain embodiments, the system may allow sustained or controlled release of the composition to occur, for example, through control of the diffusion or erosion/degradation rate of the formulation containing the CCR2 inhibitor. In addition, a pump-based hardware delivery system may be used to deliver one or more embodiments.

Examples of systems in which release occurs in bursts includes, e.g., systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be e.g., in the form of pellets, or capsules.

Use of a long-term release implant may be particularly suitable in some embodiments. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

Dosages for a particular patient can be determined by one of ordinary skill in the art using conventional considerations, (e.g., by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose can be determined by the efficacy of the particular formulation, and the activity, stability or serum half-life of the CCR2 inhibitor employed and the condition of the patient, as well as the body weight or surface area of the patient to be treated. The size of the dose can also be determined by the existence, nature, and extent of any adverse side-effects that accompany the administration of a particular composition in a particular patient.

Optimal precision in achieving concentrations of the therapeutic regimen (e.g., a pharmaceutical composition comprising one or more CCR2 inhibitor) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. Generally, the pharmaceutical compositions of the present invention may be administered in a manner that maximizes efficacy and minimizes toxicity.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See WO 00/67776, which is entirely expressly incorporated herein by reference.

More specifically, the pharmaceutical compositions may be administered in a single daily dose, or the total daily dosage may be administered in divided doses of two, three, or four times daily. In the case of oral administration, the daily dosage of the compositions may be varied over a wide range from about 0.1 ng to about 1,000 mg per patient, per day. The range may more particularly be from about 0.001 ng/kg to 10 mg/kg of body weight per day, about 0.1-100 μg, about 1.0-50 μg or about 1.0-20 mg per day for adults (at about 60 kg).

The daily dosage of the pharmaceutical compositions may be varied over a wide range from about 0.1 ng to about 1000 mg per adult human per day. For oral administration, the compositions may be provided in the form of tablets containing from about 0.1 ng to about 1000 mg of the composition or 0.1, 0.2, 0.5, 1.0, 2.0, 5.0, 10.0, 15.0, 100, 150, 200, 250, 300, 350, 400, 450, 500, 550, 600, 650, 700, 800, 900, or 1000 milligrams of the composition for the symptomatic adjustment of the dosage to the patient to be treated. An effective amount of the pharmaceutical composition is ordinarily supplied at a dosage level of from about 0.1 ng/kg to about 20 mg/kg of body weight per day. In one embodiment, the range is from about 0.2 ng/kg to about 10 mg/kg of body weight per day. In another embodiment, the range is from about 0.5 ng/kg to about 10 mg/kg of body weight per day. The pharmaceutical compositions may be administered on a regimen of about 1 to about 10 times per day.

In the case of injections, it is usually convenient to give by an intravenous route in an amount of about 0.01 μg-30 mg, about 0.01 μg-20 mg or about 0.01-10 mg per day to adults (at about 60 kg). In the case of other animals, the dose calculated for 60 kg may be administered as well.

Doses of a pharmaceutical composition described herein can optionally include 0.0001 μg to 1,000 mg/kg/administration, or 0.001 μg to 100.0 mg/kg/administration, from 0.01 μg to 10 mg/kg/administration, from 0.1 μg to 10 mg/kg/administration, including, but not limited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration or any range, value or fraction thereof.

As a non-limiting example, treatment of humans or animals can be provided as a onetime or periodic dosage of a composition described herein 0.1 ng to 100 mg/kg such as 0.0001, 0.001, 0.01, 0.1 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Specifically, the pharmaceutical compositions may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

More specifically, the pharmaceutical compositions may be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

Alternatively, the pharmaceutical compositions may be administered about once every day, about once every 2 days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days. The pharmaceutical compositions of the present invention may alternatively be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks.

Alternatively, the pharmaceutical compositions may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

Alternatively, the pharmaceutical compositions may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

Alternatively the pharmaceutical compositions may be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

Therapeutic compositions that include one or more CCR2 inhibitors can optionally be tested in one or more appropriate in vitro and/or in vivo animal models of disease, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD50 of the relevant formulation, and/or observation of any side-effects of the REV-ERBα agonist at various concentrations, e.g., as applied to the mass and overall health of the patient. Administration can be accomplished via single or divided doses.

In some embodiments, the pharmaceutical compositions including the CCR2 inhibitor can be combined with one or more therapeutic agents. In particular, the compositions described herein and other therapeutic agents can be administered simultaneously or sequentially by the same or different routes of administration. The determination of the identity and amount of therapeutic agent(s) for use in the methods described herein can be readily made by ordinarily skilled medical practitioners using standard techniques known in the art. In specific embodiments, a CCR2 inhibitor of the present invention can be administered in combination with an effective amount of a therapeutic agent that treats heart failure or nonischemic cardiomyopathy.

In other embodiments, an agent that promotes expression Kruppel-like factor 4 (KLF4) in myocardium of the subject can be administered to the subject in combination with the CCR2 inhibitor. The agent can be administered to the subject at an amount effective to enhance cardiac resident macrophage proliferation and angiogenic activities in myocardium.

In some embodiments, KLF4 can be expressed in myocardium being treated and particularly from cardiac resident macrophages using gene therapy. The gene therapy can use a vector including a nucleotide encoding KLF4. A “vector” (sometimes referred to as gene delivery or gene transfer “vehicle”) refers to a macromolecule or complex of molecules comprising a polynucleotide to be delivered to the cell. The polynucleotide to be delivered may comprise a coding sequence of interest in gene therapy. Vectors include, for example, viral vectors (such as adenoviruses (Ad), adeno-associated viruses (AAV), and retroviruses), liposomes and other lipid-containing complexes, and other macromolecular complexes capable of mediating delivery of a polynucleotide to a target cell.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities.

Vectors for use herein include viral vectors, lipid based vectors and other non-viral vectors that are capable of delivering a nucleotide encoding KLF4 described herein to the target cells. The vector can be a targeted vector, especially a targeted vector that preferentially binds to cardiac resident macrophages. Viral vectors for use in the application can include those that exhibit low toxicity to a target cell and induce expression of therapeutically useful quantities of the KLF4 in a cell specific manner.

Examples of viral vectors are those derived from adenovirus (Ad) or adeno-associated virus (AAV). Both human and non-human viral vectors can be used and the recombinant viral vector can be replication-defective in humans. Where the vector is an adenovirus, the vector can comprise a polynucleotide having a promoter operably linked to a gene encoding the therapeutic peptides and is replication-defective in humans.

Other viral vectors that can be used herein include herpes simplex virus (HSV)-based vectors. HSV vectors deleted of one or more immediate early genes (IE) are advantageous because they are generally non-cytotoxic, persist in a state similar to latency in the target cell, and afford efficient target cell transduction. Recombinant HSV vectors can incorporate approximately 30 kb of heterologous nucleic acid.

Retroviruses, such as C-type retroviruses and lentiviruses, might also be used in the application. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and a nucleic acid encoding KLF4. In methods of delivery to neural cells, it may also encode a ligand to a tissue specific receptor.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells.

Lentiviral vectors for use in the application may be derived from human and non-human (including SIV) lentiviruses. Examples of lentiviral vectors include nucleic acid sequences required for vector propagation as well as a tissue-specific promoter operably linked to a therapeutic peptide encoding nucleic acid. These former may include the viral LTRs, a primer binding site, a polypurine tract, att sites, and an encapsidation site.

A lentiviral vector may be packaged into any lentiviral capsid. The substitution of one particle protein with another from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN) might also be used in the application. Use of alphaviruses is described in Lundstrom, K., Intervirology 43:247-257, 2000 and Perri et al., Journal of Virology 74:9802-9807, 2000.

Recombinant, replication-defective alphavirus vectors are advantageous because they are capable of high-level heterologous (therapeutic) gene expression, and can infect a wide target cell range. Alphavirus replicons may be targeted to specific cell types by displaying on their virion surface a functional heterologous ligand or binding domain that would allow selective binding to target cells expressing a cognate binding partner. Alphavirus replicons may establish latency, and therefore long-term heterologous nucleic acid expression in a target cell. The replicons may also exhibit transient heterologous nucleic acid expression in the target cell.

In many of the viral vectors compatible with methods of the application, more than one promoter can be included in the vector to allow more than one heterologous gene to be expressed by the vector. Further, the vector can comprise a sequence, which encodes a signal peptide or other moiety, which facilitates expression of KLF4 from the target cell.

To combine advantageous properties of two viral vector systems, hybrid viral vectors may be used to deliver a nucleic acid encoding KLF4 to a cardiac macrophage. Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186, 2000. Retroviral genomes contained within an adenovirus may integrate within the target cell genome and effect stable gene expression.

Other nucleotide sequence elements, which facilitate expression of KLF4 and cloning of the vector are further contemplated. For example, the presence of enhancers upstream of the promoter or terminators downstream of the coding region, for example, can facilitate expression.

In accordance with another embodiment, a tissue-specific promoter can be fused to nucleotides encoding KLF4. By fusing such tissue specific promoter within the adenoviral construct, transgene expression is limited to a particular tissue. The efficacy of gene expression and degree of specificity provided by tissue specific promoters can be determined, using the recombinant adenoviral system of the present application.

In addition to viral vector-based methods, non-viral methods may also be used to introduce a nucleic acid encoding KLF4 into a target cell. A review of non-viral methods of gene delivery is provided in Nishikawa and Huang, Human Gene Ther. 12:861-870, 2001. An example of a non-viral gene delivery method according to the application employs plasmid DNA to introduce a nucleic acid encoding KLF4 into a cell. Plasmid-based gene delivery methods are generally known in the art.

Synthetic gene transfer molecules can be designed to form multimolecular aggregates with plasmid DNA. These aggregates can be designed to bind to a target cell. Cationic amphiphiles, including lipopolyamines and cationic lipids, may be used to provide receptor-independent nucleic acid transfer into target cells.

In addition, preformed cationic liposomes or cationic lipids may be mixed with plasmid DNA to generate cell-transfecting complexes. Methods involving cationic lipid formulations are reviewed in Felgner et al., Ann. N.Y. Acad. Sci. 772:126-139, 1995 and Lasic and Templeton, Adv. Drug Delivery Rev. 20:221-266, 1996. For gene delivery, DNA may also be coupled to an amphipathic cationic peptide (Fominaya et al., J. Gene Med. 2:455-464, 2000).

Methods that involve both viral and non-viral based components may be used according to the application. For example, an Epstein Barr virus (EBV)-based plasmid for therapeutic gene delivery is described in Cui et al., Gene Therapy 8:1508-1513, 2001. Additionally, a method involving a DNA/ligand/polycationic adjunct coupled to an adenovirus is described in Curiel, D. T., Nat. Immun. 13:141-164, 1994.

Additionally, the nucleic acid encoding KLF4 can be introduced into the target cell by transfecting the target cells using electroporation techniques. Electroporation techniques are well known and can be used to facilitate transfection of cells using plasmid DNA.

Vectors that encode the expression of KLF4 can be delivered in vivo to the target cell in the form of an injectable preparation containing pharmaceutically acceptable carrier, such as saline, as necessary. Other pharmaceutical carriers, formulations and dosages can also be used in accordance with the present application.

KLF4 can be expressed for any suitable length of time within the target cell, including transient expression and stable, long-term expression. In one aspect of the application, the nucleic acid encoding KLF4 will be expressed in therapeutic amounts for a defined length of time effective to induce activity and growth of the transfected cells. In another aspect of the application, the nucleic acid encoding KLF4 will be expressed in therapeutic amounts for a defined length of time effective to enhance cardiac resident macrophage proliferation and angiogenic activities.

In some embodiments, the KLF4 can be administered to a weakened region, or hypertrophic region of the myocardial tissue by direct injection of the vector encoding KLF4 into or about the periphery the weakened region, or hypertrophic region of the myocardial tissue at an amount effective to enhance cardiac resident macrophage proliferation and angiogenic activities, reduce arrhythmic susceptibility, and/or normalize electrophysiology of the myocardial tissue. By injecting the therapeutic agent directly into or about the periphery of the weakened or hypertrophic region of the myocardial tissue, it is possible to target the therapeutic agent to the tissue. This can enable local delivery to and/or transduction of a desired number of cells, especially about the weakened or hypertrophic region of the myocardial tissue, thereby maximizing therapeutic efficacy of protein transduction or gene transfer, and minimizing the possibility of an inflammatory response.

It will be appreciated that the amount, volume, concentration, and/or dosage of the agent that is administered to any one animal or human depends on many factors, including the subject's size, body surface area, age, the particular composition to be administered, sex, time and route of administration, general health, and other drugs being administered concurrently. Specific variations of the above noted amounts, volumes, concentrations, and/or dosages of therapeutic agent can be readily be determined by one skilled in the art using the experimental methods described below.

In some embodiments, the therapeutic agent can be administered by direct injection using catheterization, such as endo-ventricular catheterization or intra-myocardial catheterization. In one example, a deflectable guide catheter device can be advanced to a left ventricle retrograde across the aortic valve. Once the device is positioned in the left ventricle, the therapeutic agent can be injected into the peri-infarct region (both septal and lateral aspect) area of the left ventricle.

The myocardial tissue of the subject can be imaged prior to administration of the therapeutic agent to define the area of weakened or hypertrophic region prior to administration of the therapeutic agent. Defining the weakened or hypertrophic region by imaging allows for more accurate intervention and targeting of the therapeutic agent to the weakened or hypertrophic region. The imaging technique used to define the weakened or hypertrohic region of the myocardial tissue can include any known cardio-imaging technique. Such imaging techniques can include, for example, at least one of echocardiography, magnetic resonance imaging, coronary angiogram, electroanatomical mapping, or fluoroscopy. It will be appreciated that other imaging techniques that can define the weakened, ischemic, and/or peri-infarct region can also be used.

In another embodiment, the therapeutic agent can be administered to a subject systemically by intravenous injection or locally at the site of injury, usually within about 24 hours, about 48 hours, about 100 hours, or about 200 hours or more of when an injury occurs (e.g., within about 6 hours, about 12 hours, or 24 hours, inclusive, of the time of the injury).

In some embodiments, the therapeutic agent can administered to a subject for an extended period of time to inhibit, ameliorate, and/or reduce arrhythmias in the heart of the subject. Sustained contact with the therapeutic agent can be achieved, for example, by repeated administration of the active compound(s) over a period of time, such as one week, several weeks, one month or longer.

In other embodiments, a pharmaceutically acceptable formulation used to administer the therapeutic agent(s) can also be formulated to provide sustained delivery of the active compound to a subject. For example, the formulation may deliver the active compound for at least one, two, three, or four weeks, inclusive, following initial administration to the subject. For example, a subject to be treated in accordance with the method described herein can be treated with the therapeutic agent for at least 30 days (either by repeated administration or by use of a sustained delivery system, or both).

Sustained delivery of the therapeutic agent can be demonstrated by, for example, the continued therapeutic effect of the therapeutic agent over time (such as sustained delivery of the agents can be demonstrated by continued inhibitions of arrhythmias in a subject). Alternatively, sustained delivery of the therapeutic agent may be demonstrated by detecting the presence of the therapeutic agents in vivo over time.

Approaches for sustained delivery include use of a polymeric capsule, a minipump to deliver the formulation, a biodegradable implant, or implanted transgenic autologous cells (see U.S. Pat. No. 6,214,622). Implantable infusion pump systems (e.g., INFUSAID pumps (Towanda, Pa.)); see Zierski et al., 1988; Kanoff, 1994) and osmotic pumps (sold by Alza Corporation) are available commercially and otherwise known in the art. Another mode of administration is via an implantable, externally programmable infusion pump. Infusion pump systems and reservoir systems are also described in, e.g., U.S. Pat. Nos. 5,368,562 and 4,731,058.

Vectors encoding the therapeutic peptides can often be administered less frequently than other types of therapeutics. For example, an effective amount of such a vector can range from about 0.01 mg/kg to about 5 or 10 mg/kg, inclusive; administered daily, weekly, biweekly, monthly or less frequently.

In some embodiment, the efficacy of the KLF4 in treating cardiomyopathy and/or heart failure of a subject in need thereof can be measured using, for example, electrocardiogram (ECG) monitoring to determine the electrophysiology of the heart. The ECG measurements can be compared to normal or control ECG measurements to determine efficacy of the therapeutic agent in normalizing or restoring electrophysiology of the heart. In some embodiments, ECG can be used in a conjunction with a cardiac stress test or with telemetry to determine efficacy of therapeutic agent in promoting cardiac resident macrophage proliferation and angiogenesis in the heart.

It will be appreciated that other agents can be administered in combinations with the CCR2 inhibitor and/or the agent that promotes KLF4 expression. These other therapeutic agents can include, but are not limited to, beta blockers, anti-hypertensives, cardiotonics, anti-thrombotics, vasodilators, hormone antagonists, inotropes, diuretics, endothelin antagonists, calcium channel blockers, phosphodiesterase inhibitors, ACE inhibitors, angiotensin type 2 antagonists and cytokine blockers/inhibitors, and HDAC inhibitors.

More specifically the CCR2 inhibitor and/or the agent that promotes KLF4 expression may be combined with another therapeutic agent including, but not limited to, an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

In specific embodiments, the CCR2 inhibitor and/or the agent that promotes KLF4 expression may be combined with an antihyperlipoproteinemic agent including aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof, acifran, azacosterol, benfluorex, β-benzalbutyramide, carnitine, chondroitin sulfate, clomestrone, detaxtran, dextran sulfate sodium, eritadenine, furazabol, meglutol, melinamide, mytatrienediol, ornithine, γ-oryzanol, pantethine, pentaerythritol tetraacetate, phenylbutyramide, pirozadil, probucol (lorelco), β-sitosterol, sultosilic acid-piperazine salt, tiadenol, triparanol and xenbucin.

The CCR2 inhibitor and/or the agent that promotes KLF4 expression may be combined with an antiarteriosclerotic agent such as pyridinol carbamate. In other embodiments, a REV-ERBα agonist may be combined with an antithrombotic/fibrinolytic agent including, but not limited to anticoagulants (acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin); anticoagulant antagonists, antiplatelet agents (aspirin, a dextran, dipyridamole (persantin), heparin, sulfinpyranone (anturane) and ticlopidine (ticlid)); thrombolytic agents (tissue plaminogen activator (activase), plasmin, pro-urokinase, urokinase (abbokinase) streptokinase (streptase), anistreplase/APSAC (eminase)); thrombolytic agent antagonists or combinations thereof).

In other embodiments, the CCR2 inhibitor and/or the agent that promotes KLF4 expression may be combined with a blood coagulant including, but not limited to, thrombolytic agent antagonists (amiocaproic acid (amicar) and tranexamic acid (amstat); antithrombotics (anagrelide, argatroban, cilstazol, daltroban, defibrotide, enoxaparin, fraxiparine, indobufen, lamoparan, ozagrel, picotamide, plafibride, tedelparin, ticlopidine and triflusal); and anticoagulant antagonists (protamine and vitamine K1).

Alternatively, the CCR2 inhibitor and/or the agent that promotes KLF4 expression may be combined with an antiarrhythmic agent including, but not limited to, Class I antiarrythmic agents (sodium channel blockers), Class II antiarrythmic agents (beta-adrenergic blockers), Class II antiarrythmic agents (repolarization prolonging drugs), Class IV antiarrhythmic agents (calcium channel blockers) and miscellaneous antiarrythmic agents. Non-limiting examples of sodium channel blockers include Class IA (disppyramide (norpace), procainamide (pronestyl) and quinidine (quinidex)); Class IB (lidocaine (xylocalne), tocamide (tonocard) and mexiletine (mexitil)); and Class IC antiarrhythmic agents, (encamide (enkaid) and fiecamide (tambocor)).

Non-limiting examples of a beta blocker (also known as a β-adrenergic blocker, a β-adrenergic antagonist or a Class II antiarrhythmic agent) include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfmalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol. Non-limiting examples of an agent that prolongs repolarization, also known as a Class III antiarrhythmic agent, include amiodarone (cordarone) and sotalol (betapace).

Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexyline. In certain embodiments a calcium channel blocker comprises a long-acting dihydropyridine (nifedipine-type) calcium antagonist.

Non-limiting examples of miscellaneous antiarrhymic agents include adenosine (adenocard), digoxin (lanoxin), acecamide, ajmaline, amoproxan, aprindine, bretylium tosylate, bunaftine, butobendine, capobenic acid, cifenline, disopyranide, hydroquinidine, indecamide, ipatropium bromide, lidocaine, lorajmine, lorcamide, meobentine, moricizine, pirmenol, prajmaline, propafenone, pyrinoline, quinidine polygalacturonate, quinidine sulfate and viquidil.

In other embodiments, a REV-ERBα agonist may be combined with an antihypertensive agent including, but not limited to, alpha/beta blockers (labetalol (normodyne, trandate)), alpha blockers, anti-angiotensin II agents, sympatholytics, beta blockers, calcium channel blockers, vasodilators and miscellaneous antihypertensives.

Non-limiting examples of an alpha blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

Non-limiting examples of anti-angiotension II agents include angiotensin converting enzyme inhibitors and angiotension II receptor antagonists. Non-limiting examples of angiotensin converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan. Non-limiting examples of a sympatholytic include a centrally acting sympatholytic or a peripherally acting sympatholytic.

Non-limiting examples of a centrally acting sympatholytic, also known as a central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or an α1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).

In certain embodiments, an antihypertensive agent may comprise a vasodilator (e.g., a cerebral vasodilator, a coronary vasodilator or a peripheral vasodilator). In particular embodiments, a vasodilator comprises a coronary vasodilator including, but not limited to, amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(P-diethylaminoethyl ether), hexobendine, itramin tosylate, khellin, lidoflanine, mannitol hexanitrane, medibazine, nicorglycerin, pentaerythritol tetranitrate, pentrinitrol, perhexyline, pimethylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil. In certain aspects, an antihypertensive may comprise an arylethanolamine derivative (amosulalol, bufuralol, dilevalol, labetalol, pronethalol, sotalol and sulfmalol); a benzothiadiazine derivative (althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlonnethiazide); a N-carboxyalkyl(peptide/lactam) derivative (alacepril, captopril, cilazapril, delapril, enalapril, enalaprilat, fosinopril, lisinopril, moveltipril, perindopril, quinapril and ramipril); a dihydropyridine derivative (amlodipine, felodipine, isradipine, nicardipine, nifedipine, nilvadipine, nisoldipine and nitrendipine); a guanidine derivative (bethanidine, debrisoquin, guanabenz, guanacline, guanadrel, guanazodine, guanethidine, guanfacine, guanochlor, guanoxabenz and guanoxan); a hydrazines/phthalazine (budralazine, cadralazine, dihydralazine, endralazine, hydracarbazine, hydralazine, pheniprazine, pildralazine and todralazine); an imidazole derivative (clonidine, lofexidine, phentolamine, tiamenidine and tolonidine); a quanternary ammonium compound (azamethonium bromide, chlorisondamine chloride, hexamethonium, pentacynium bis(methylsulfate), pentamethonium bromide, pentolinium tartrate, phenactropinium chloride and trimethidinium methosulfate); a reserpine derivative (bietaserpine, deserpidine, rescinnamine, reserpine and syrosingopine); or a sulfonamide derivative (ambuside, clopamide, faro semide, indapamide, quinethazone, tripamide and xipamide).

In other embodiments, the CCR2 inhibitor and/or the agent that promotes KLF4 expression may be combined with a vasopressor. Vasopressors generally are used to increase blood pressure during shock, which may occur during a surgical procedure. Non-limiting examples of a vasopressor, also known as an antihypotensive include amezinium methyl sulfate, angiotensin amide, dimetofrine, dopamine, etifelmin, etilefrin, gepefrine, metaraminol, midodrine, norepinephrine, pholedrine and synephrine.

the CCR2 inhibitor and/or the agent that promotes KLF4 expression may be combined with treatment agents for congestive heart failure including, but not limited to, anti-angiotension II agents, afterload-preload reduction treatment (hydralazine (apresoline) and isosorbide dinitrate (isordil, sorbitrate)), diuretics, and inotropic agents.

Non-limiting examples of a diuretic include a thiazide or benzothiadiazine derivative (e.g., althiazide, bendroflumethazide, beizthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexyline, ticrnafen and urea.

Non-limiting examples of a positive inotropic agent, also known as a cardiotonic, include acefylline, an acetyldigitoxin, 2-amino-4-picoline, aminone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include aminone (inocor).

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for hypertrophy, vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

Alternatively, therapeutic agents that can be administered in combination therapy with one or more of the CCR2 inhibitor and/or the agent that promotes KLF4 expression include, but are not limited to, anti-inflammatory, anti-viral, anti-fungal, anti-mycobacterial, antibiotic, amoebicidal, trichomonocidal, analgesic, anti-neoplastic, anti-hypertensives, anti-microbial and/or steroid drugs, to treat cardiac hypertrophy and/or any heart disease associated with cardiac hypertrophy. In some embodiments, patients are treated with a REV-ERBα agonist in combination with one or more of the following; β-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, sulindac, acyclovir, amantadine, rimantadine, recombinant soluble CD4 (rsCD4), anti-receptor antibodies (e.g., for rhinoviruses), nevirapine, cidofovir (Vistide™) trisodium phosphonoformate (Foscarnet™), famcyclovir, pencyclovir, valacyclovir, nucleic acid/replication inhibitors, interferon, zidovudine (AZT, Retrovir™), didanosine (dideoxyinosine, ddl, Videx™), stavudine (d4T, Zerit™), zalcitabine (dideoxycytosine, ddC, Hivid™), nevirapine (Viramune™), lamivudine (Epivir™, 3TC), pro tease inhibitors, saquinavir (Invirase™, Fortovase™), ritonavir (Norvir™), nelfmavir (Viracept™), efavirenz (Sustiva™) abacavir (Ziagent™), amprenavir (Agenerase™) indinavir (Crixivan™), ganciclovir, AzDU, delavirdine (Kescriptor™), kaletra, trizivir, rifampin, clathiromycin, erythropoietin, colony stimulating factors (G-CSF and GM-CSF), non-nucleoside reverse transcriptase inhibitors, nucleoside inhibitors, adriamycin, fluorouracil, methotrexate, asparagyinase and combinations foregoing.

In another aspect, the CCR2 inhibitor and/or the agent that promotes KLF4 expression may be combined with other therapeutic agents including, but not limited to, immunomodulatory agents, anti-inflammatory agents (e.g., adrenocorticoids, corticosteroids (e.g., beclomethasone, budesonide, flunisolide, fluticasone, triamcinolone, methlyprednisolone, prednisolone, prednisone, hydrocortisone), glucocorticoids, steroids, non-steriodal antiinflammatory drugs (e.g., aspirin, ibuprofen, diclofenac, and COX-2 inhibitors), and leukotreine antagonists (e.g., montelukast, methyl xanthines, zafirlukast, and zileuton), β2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), other anti-viral agents, and antibiotics (e.g., dactinomycin (formerly actinomycin), bleomycin, erythomycin, penicillin, mithramycin, and anthramycin (AMC)).

In various embodiments, the CCR2 inhibitor and/or the agent that promotes KLF4 expression in combination with a second therapeutic agent may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In particular embodiments, two or more therapies are administered within the same patent visit.

In certain embodiments, the CCR2 inhibitor and/or the agent that promotes KLF4 expression and one or more other therapies are cyclically administered. Cycling therapy involves the administration of a first therapy (e.g., a REV-ERBα agonist) for a period of time, followed by the administration of a second therapy (e.g., a second REV-ERBα agonist or another therapeutic agent) for a period of time, optionally, followed by the administration of a third therapy for a period of time and so forth, and repeating this sequential administration, e.g., the cycle, in order to reduce the development of resistance to one of the therapies, to avoid or reduce the side effects of one of the therapies, and/or to improve the efficacy of the therapies. In certain embodiments, the administration of the combination therapy of the present invention may be repeated and the administrations may be separated by at least 1 day, 2 days, 3 days, 5 days, 10 days, 15 days, 30 days, 45 days, 2 months, 75 days, 3 months, or at least 6 months.

The dosages of a pharmaceutical composition disclosed herein may be adjusted when combined to achieve desired effects. On the other hand, dosages of the pharmaceutical composition and various therapeutic agents may be independently optimized and combined to achieve a synergistic result wherein the pathology is reduced more than it would be if either were used alone.

The following example is included to demonstrate different embodiments of the invention. It should be appreciated by those of skill in the art that the techniques disclosed in the example, which follow represent techniques discovered by the inventors to function well in the practice of the claimed embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the claims.

### Example

Using a classic mouse pressure overload hypertrophy (POH) model for nonischemic cardiomyopathy (NICM), we tracked resident and infiltrating macrophages in the myocardium during the early and late phases of POH and show previously unappreciated and distinct time dependent roles of different subsets of cardiac macrophages in POH. Using the myeloid-specific Kruppel-like factor 4 (KLF4) mutant mouse, we also shed light on the molecular mechanism that regulates local proliferation of cardiac resident macrophages and a potential role that these cells play in regulating perfusion during POH.

### Materials and Methods

### Cell Culture

Mouse thioglycollate-elicited peritoneal macrophages (PM) and mouse bone marrow derived macrophages (BMDM) were prepared as described previously.

### Animals

Mice with myeloid-specific deficiency of Klf4 (Lyz2-Cre:Klf4fl/fl, K4-cKO) or IL4Ra (Lyz2-Cre:IL4Rafl/fl, IL4Ra-cKO) were described previously. The Lyz2-Cre line (Cre) was used as genetic control. WT C57BL/6J (B6-CD45.2), B6-CD45.1 and CCR2-KO mice were purchased from Jackson laboratory. All mice are on a C57BL/6J background. Mice were housed in a temperature- and humidity-controlled specific pathogen-free facility with a 12-hour-light/dark cycle and ad libitum access to water and standard laboratory rodent chow.

### CD45.1-CD45.2 Chimera Mice

Partial bone marrow transplantation was performed with B6-CD45.2 as recipient and B6-CD45.1 as donor. The rear part of recipient CD45.2 mice were exposed to lethal dose gamma ray while chest and head area was shielded by lead block. After irradiation, two million CD45.1 bone marrow cells were transplanted via tail vein injection and recipients were allowed to recover for 2 months before blood test for CD45.1 cells. Blood (50 uL) was drawn from tail and subjected to immunostaining with fluorescent antibodies for CD11b, CD45.1, and CD45.2 for FACS analysis. Mice with >50% CD45.1 myeloid cells were used for TAC models.

### Macrophage Depletion, CCR2 Blockade and Neutrophil Depletion

Macrophage depletion was achieved by injection (i.v.) of Clodronate liposomes at 500 ug/kg on every other day, starting from 1-day before or 14-day after cardiac surgery as indicated in specific experiments. PBS-containing liposomes were used as vehicle control (Veh). Clodronate liposomes and control liposomes were obtained from the nonprofit Consortium Foundation Clodronate Liposomes. To inhibit monocyte trafficking in vivo, RS-504393 was dissolved in DMSO, diluted 1:20 in PBS and administrated at 2 mg/kg twice daily by oral gavage, starting from 1-3 day before or 14-day after cardiac surgery as indicated in specific experiments. PBS with 5% DMSO was used as vehicle control.

Neutrophil depletion was achieved by injection (i.p.) of anti-Ly6G antibody (clone 1A8, Bio X Cell). Total 100 ug antibody in 100 ul PBS was injected per mouse (body weight 25-30 g). To maintain stable depletion, mice were injected every other day. Normal rat IgG was used as control.

### Pressure Overload Hypertrophy Studies

Transverse aortic constriction (TAC) model and echocardiography were performed as described previously. Mice were anaesthetized by ketamine xylazine cocktail (100 uL i.p.) for TAC surgery to reduce aorta diameter to 27-gauge. Buprenorphine was administrated daily (i.p.) for first 5-days post-TAC. Blood pressure gradient across ligation site was confirmed by Doppler echography. For echocardiography, mice were anaesthetized by inhalation of 1% isoflurane vaporized in 100% oxygen. LV function was recorded at parasternal short axis view in B-mode and M-mode on a VEVO 770 High-Resolution In Vivo Micro-Imaging System (VisualSonics). ECHO data were recorded at steady heart rate above 500 bpm. LV function was calculated as ejection fraction (EF) or fractional shortening (FS).

### Flow Cytometry

Peritoneal macrophages were collected as described previously. Cardiac macrophages were gated from whole heart cells using surface antibodies (CD45+CD11b+F4/80+Ly6G-CD64+). Mouse heart is perfused, excised, minced, digested with Type I collagenase, mechanically disrupted and filtered through 70 μm cell strainer to get single cell suspension. Cells are collected by centrifugation, subjected to live-death dye and surface antibody staining, and analyzed by flow cytometry. Cells were also sorted by flow cytometry to collect cardiac macrophages. For Ki-67 staining, surface stained cells were fixed and permeabilized by the FIX PERM Cell Permeabilization Kit (ThermoFisher), and further stained with anti-Ki-67 antibody. DNA was stained with Hoechst 33342. For monocyte analysis, blood was collected from inferior vena cava (IVC), bone marrow cells were flush from femurs, and spleen was mashed on a 40 μm cell strainer. After RBC lysis, cells were stained with surface antibodies and analyzed by FACS. Monocytes were gated as CD115+CD11b+ population and further gated for CX3CR1, Ly6C and CCR2 to distinguish classic vs. -non-classic monocytes.

### In Vivo BrdU Pulse Labeling Studies

To assess local proliferation of cardiac resident macrophages, animals were injected (i.p.) with 2 mg BrdU and pulse labelled for 2 hours before harvest. During this 2-hour period, BrdU-labeled bone marrow cells have not been released into circulation, so that only local proliferating cells will be labeled as BrdU positive. The hearts were digested to single cell suspension and stained for cell surface markers as above. The BD Pharmingen™ APC BrdU Flow Kit (BD Sciences, 552598) was used to detect BrdU-labelled cells. In brief, surface stained cells were fixed and permeabilized using BD Cytofix/Cytoperm buffer and Cytoperm Permeabilization Plus buffer; digested with DNase I to expose BrdU antigen, stained with fluorescent APC-conjugated anti-BrdU antibody and analyzed using a BD LSR-II flow cytometer. DNA was stained with Hoechst 33342 for cycle analysis.

### Histology

Cardiac tissue samples were fixed in 10% neutralized formalin and embedded with paraffin following standard protocol. Fibrosis was visualized using Picrosirius Red Stain Kit (Polysciences, Inc.) or Gomori's Trichrome staining kit (Sigma). To determine cell death in the myocardium, TUNEL staining was performed with the Peroxidase-based or Fluorescein-based ApopTag In Situ Apoptosis Detection Kit (Millipore, 57100, 57110) following manufacturer's protocol. In fluorescent TUNEL staining, heart sections were double stained with anti-cardiac-actin (Sigma, A9357) primary antibody and Alexa Fluor 594-conjugated goat-anti-mouse-IgG secondary antibody (ThermoFisher) to visualize cardiomyocytes. To determine apoptosis, paraffinembedded heart sections were stained with anti-Cleaved Caspase-3 (Asp175) antibody using the SignalStain® Apoptosis (Cleaved Caspase-3) IHC Detection Kit (Cell Signaling Technology, 12692S). Capillary staining was performed in cryopreserved heart sections using anti-CD31 primary antibody (1:50, BD 553370) and Alexa Fluor 594-conjugated goat-anti-rat-IgG secondary antibody (ThermoFisher). Cardiomyocyte cross sectional area was determined by staining with Alexa Fluor 5904-conjugated Wheat Germ Agglutinin (WGA) (ThermoFisher). Microscopic images were analyzed using NIH ImageJ software for quantification and channel overlay.

### Transmission Electron Microscopy

Small pieces of tissue from the LV free wall were fixed by sequential immersion in triple aldehyde5 DMSO, ferrocyanide-reduced osmium tetroxide, and acidified uranyl acetate; dehydrated in ascending concentrations of ethanol; passed through propylene oxide; and embedded in Poly/Bed resin (Polysciences Inc., 21844-1). Thin sections were sequentially stained with acidified uranyl acetate, followed by a modification of Sato's triple lead stain, and examined with a JEOL 1200EX electron microscope.

RNA Extraction and qPCR

Tissue samples were homogenized in QIAzol lysis reagent (Qiagen, 79306) with a TissueLyser (Qiagen). Cell samples were directly dissolved in QIAzol lysis reagent. Total RNA was extracted, treated with DNase I (Life Technologies, 18068015), purified using the RNeasy MinElute Cleanup Kit (Qiagen, 74204). The Qiagen RNeasy Micro Kit was used to extract RNA from small amount of FACS-sorted cells (5,000-10,000 cells). RNA was reverse transcribed to complementary DNA using the iScript Reverse Transcription Kit (Bio-Rad, 170-8841). Quantitative real-time PCR (qPCR) was performed with either the TaqMan method (Roche Universal ProbeLibrary System) or the SYBR green method on a ViiA 7 Real-Time PCR System (Applied Biosystems). Relative expression was calculated using the ΔΔCt method with normalization to Gapdh.

### RNA-Seq Studies

For RNASeq, libraries were prepared using the Illumina TruSeq Stranded Total RNA Sample Preparation kit according to the manufacturer's protocol. 50 bp singled-end sequencing was performed on pooled libraries in groups of three using an Illumina HiSeq 2500. Sequencing reads generated from the Illumina platform were assessed for quality using FastQC. The reads were then trimmed for adapter sequences using TrimGalore. For RNASeq, reads that passed quality control were then aligned to rn6 using TOPHAT. The TOPHAT results were then analyzed for differential expression using cufflinks to generate the fragments per kilobase of exon per million fragments mapped (FPKM) for each gene. Differential genes were identified using a significance cutoff of q<0.005 and fold change>1.5. These genes were then subjected to further analysis. Gene Ontology (GO) Enrichment Analysis was performed with the DAVID Bioinformatics Resources 6.8. RNA-seq data are deposited at Gene Expression Omnibus (GSE107016).

### Results

### Pressure Overload Induces Local Proliferation of Cardiac Resident Macrophages.

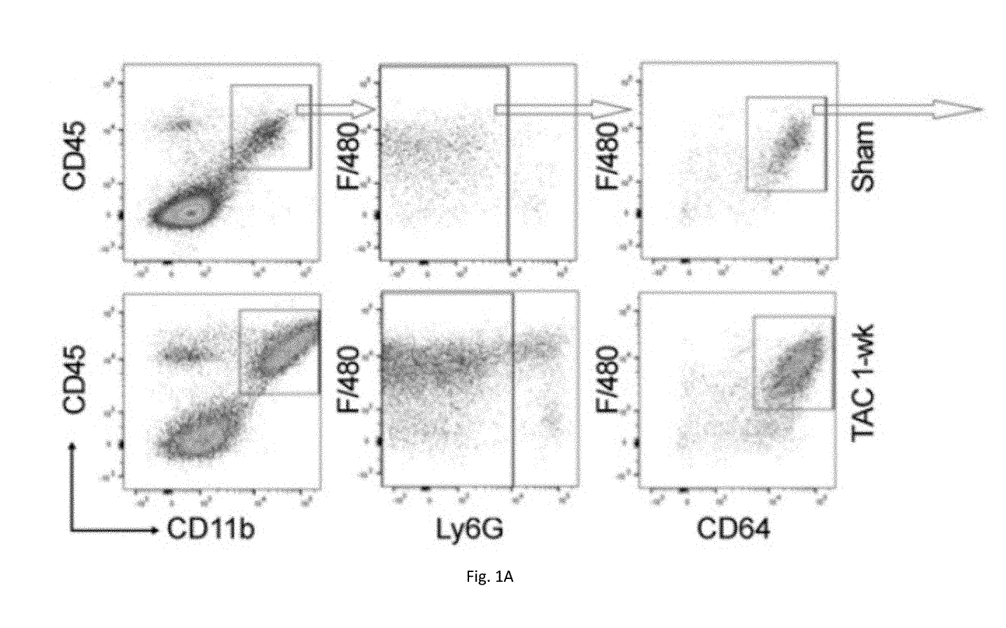

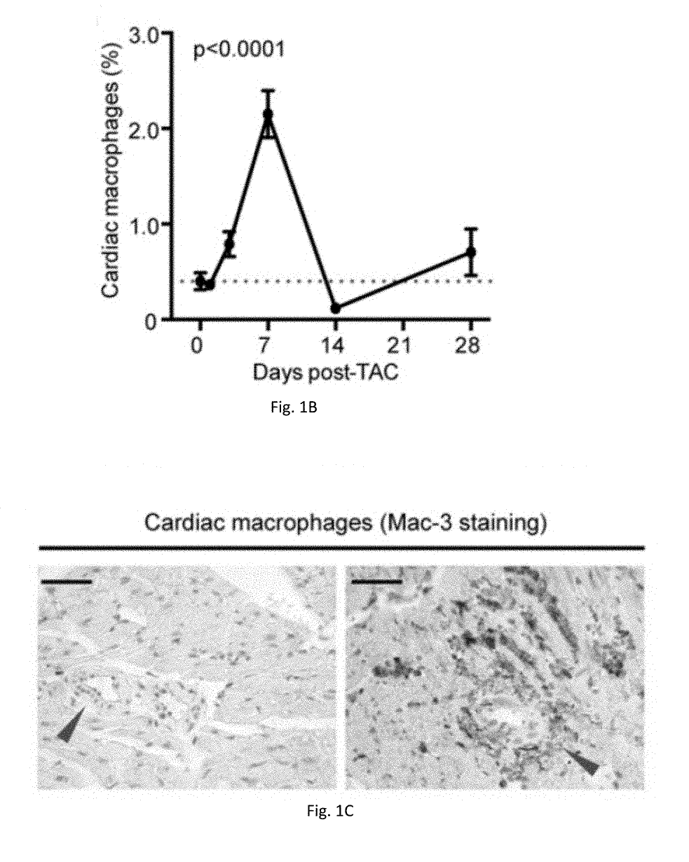

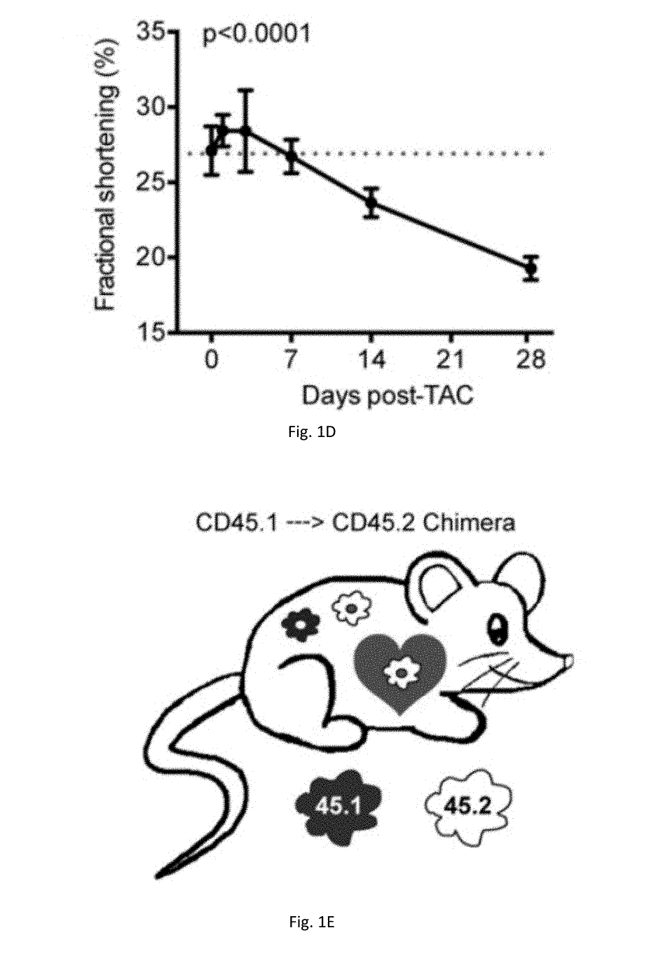

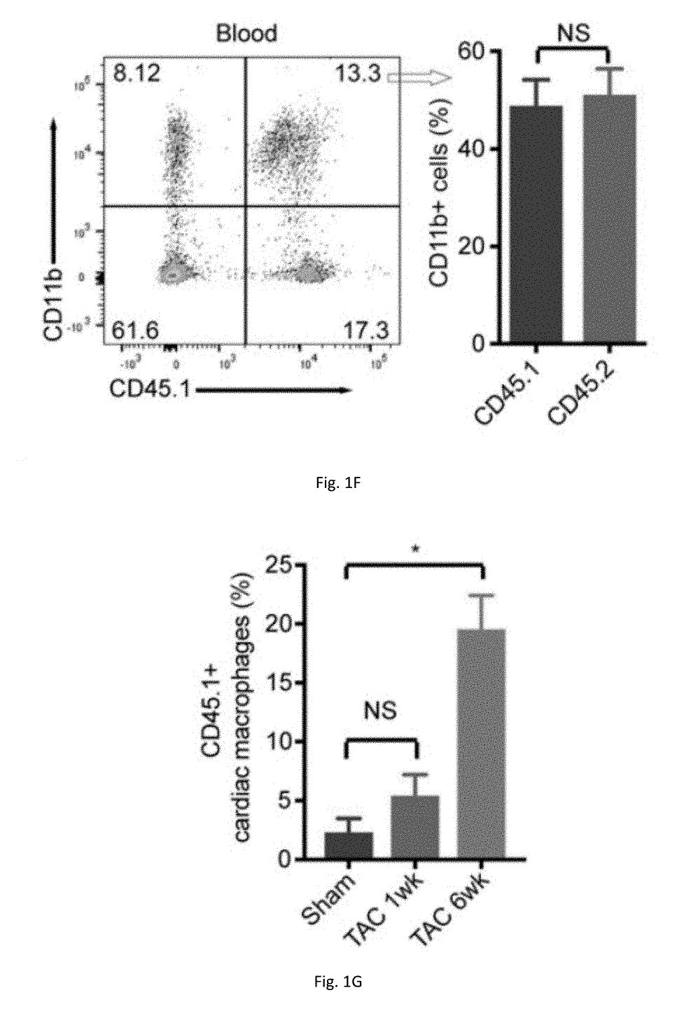

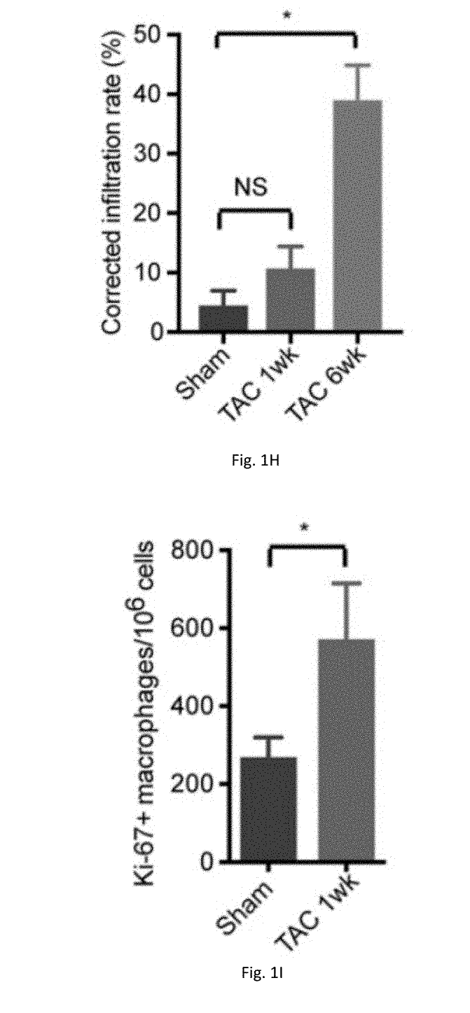

To understand the role of macrophages in POH, we first assessed macrophage numbers in the heart following transverse aortic constriction (TAC), a well-established model of pressure overload-induced hypertrophy and heart failure (i.e., POH). Hearts from sham or TAC animals were analyzed by flow cytometry. As shown in FIG. 1A, we found that TAC induced significant accumulation of myeloid cells (CD45+/CD11b+) in the myocardium. The majority of these cells are macrophages (Ly6G−/F4/80+/CD64+ subset; >95%), and the remainder are Ly6G+ neutrophils (<5%) (FIG. 1A). Time course studies showed that cardiac macrophages increased significantly by 3 d post-TAC, peaked at 7 d, returned to baseline after 2 wk, and then increased modestly at 4 wk after TAC (FIG. 1B). The presence of macrophages in the myocardium was further confirmed by anti-Mac-3 immunostaining, particularly in the perivascular region (FIG. 1C). This dynamic change in cardiac macrophage numbers is noteworthy as it correlates with two major phases of the cardiac response to TAC, namely, the compensatory cardiac hypertrophy with preserved contractile function phase that characterizes the first 7-10 d following TAC, followed by decompensation and development of heart failure between 2 and 4 wk after TAC (FIG. 1D).

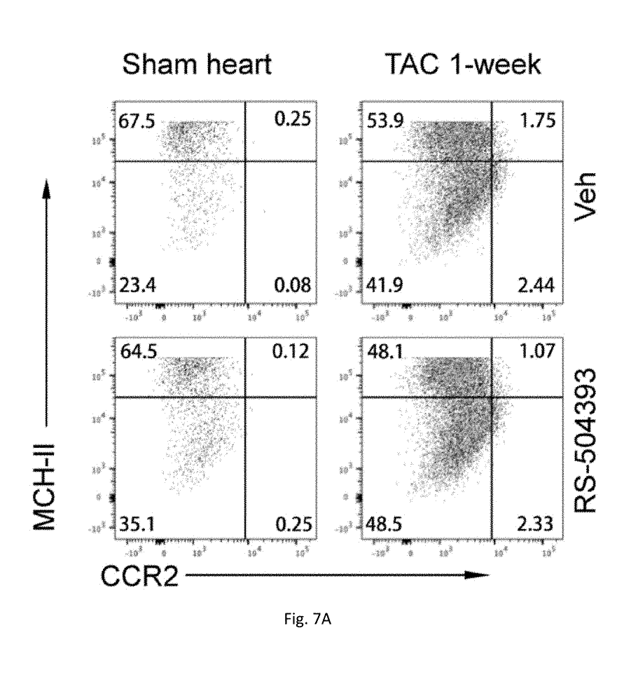

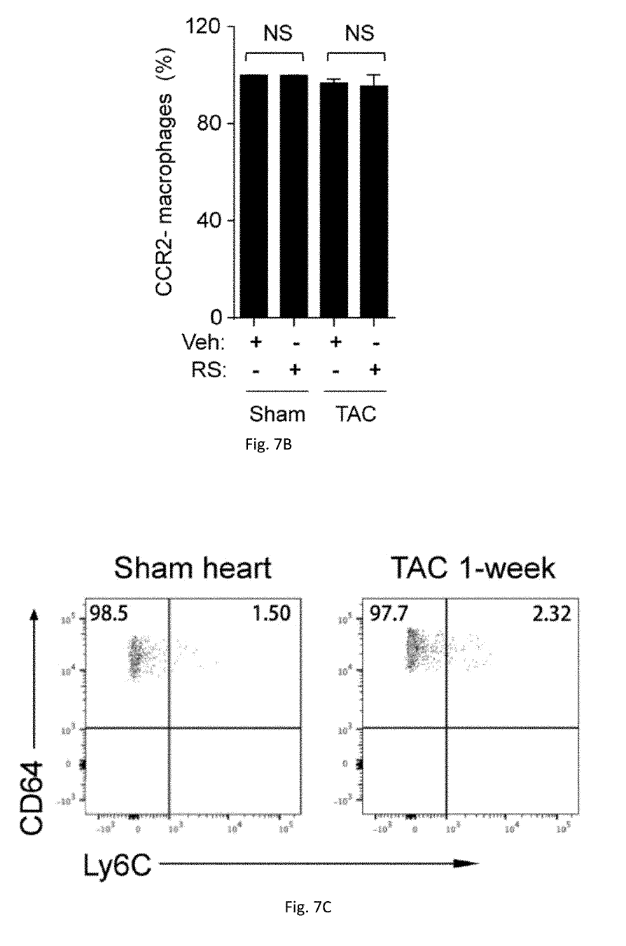

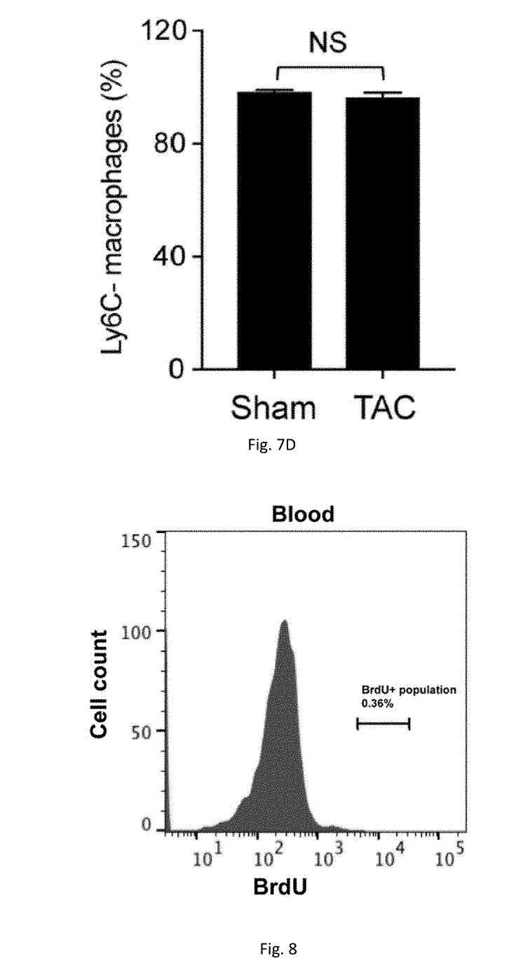

Next, we undertook multiple complementary approaches to determine if the increase in cardiac macrophages following TAC was from resident macrophages, blood monocytes recruited to the myocardium, or both. First, we utilized a recently described fluorescence-activated cell sorting (FACS) strategy in which the surface expression of CCR2 is low in resident macrophages but high in infiltrating macrophages. Importantly, we found that macrophages accumulating after 1-wk TAC are CCR2−, indicating that they are resident macrophages (FIGS. 7A and B). Second, we showed that blockade of monocyte infiltration by RS-504393 (RS), a small-molecule CCR2 antagonist, had no effect on cardiac macrophage populations (FIGS. 7A and B). Consistently, most cardiac macrophages are Ly6Clo before and after 1-wk TAC (FIGS. 7C and D). Finally, to validate if pressure overload only induces resident macrophage expansion, we utilized a CD45.1-CD45.2 chimeric mouse model, in which recipient CD45.2 mice were irradiated utilizing a thorax shield to minimize cardiac macrophage depletion. Following transplantation, the donor CD45.1⁺ bone marrow cells replaced circulating CD45.2+ blood cells without affecting cardiac resident macrophages (FIG. 1E). We chose the chimeric animals with ˜50% CD45.1⁺ myeloid cells (CD11b⁺) for the TAC model to ensure effective in vivo cell tracking (FIG. 1F). In sham animals, we observed that only 2.3% of cardiac macrophages are CD45.1+, confirming that cardiac macrophages are maintained locally at steady state (FIG. 1G). After 1-wk TAC, the CD45.1⁺ cardiac macrophages remained low at 5.4%, suggesting that no significant monocyte infiltration occurs at this early stage of POH (FIG. 1G). However, after 6-wk TAC, the CD45.1+ cardiac macrophages reached 20% (FIG. 1G), indicating monocyte infiltration in late-stage POH that is associated with the transition from cardiac compensation to decompensation. Because the CD45.1-CD45.2 chimeras subjected to TAC carried both CD45.1⁺ and CD45.2⁺ white blood cells in circulation (1:1 by experimental design), the above percentages of CD45.1+ macrophages only reflect 50% of the actual infiltrating cells. As such, the corrected rate of monocyte infiltration into the myocardium is about 5-10% at baseline and early-phase POH, and it increases to around 40% in late-phase POH (FIG. 1H).

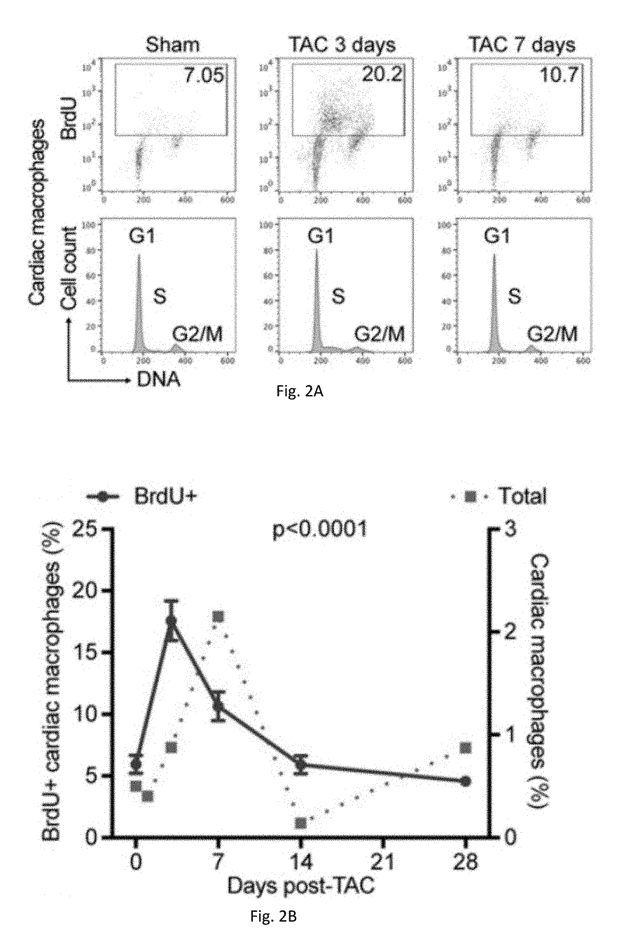

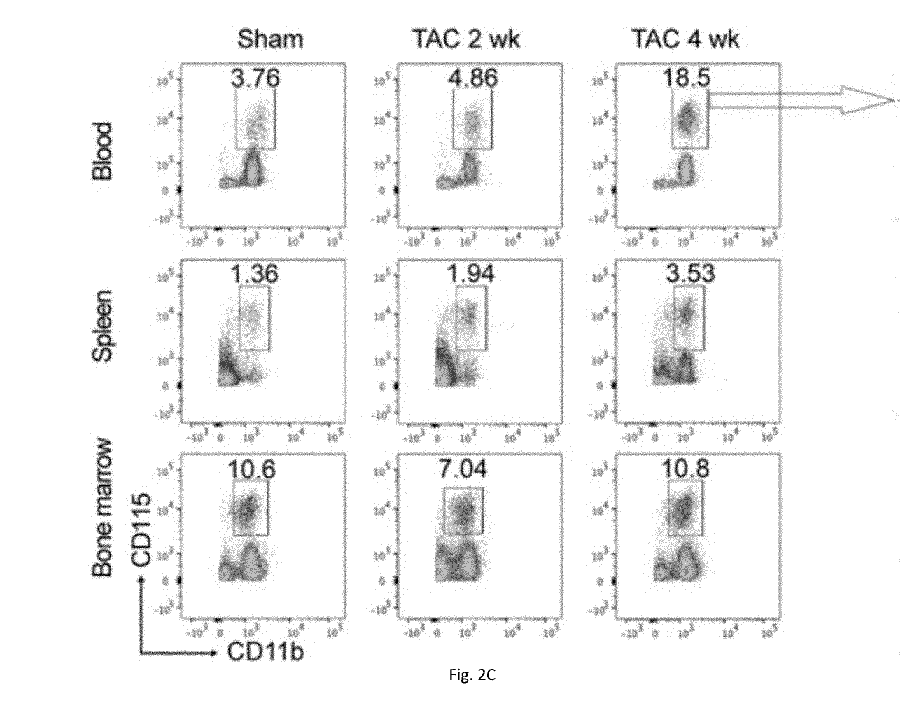

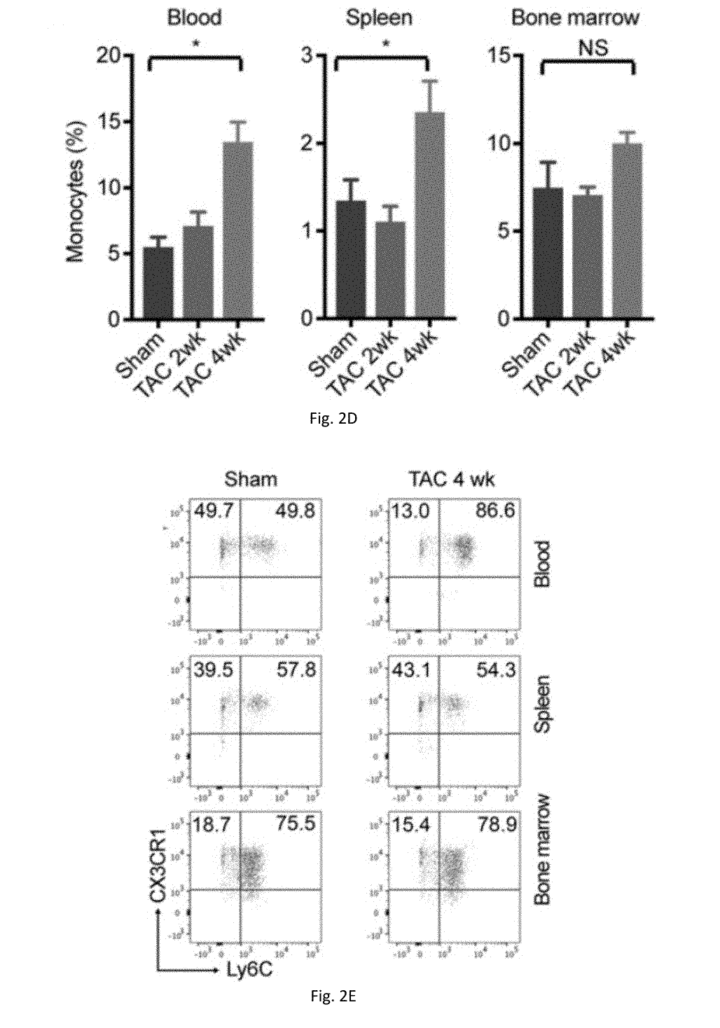

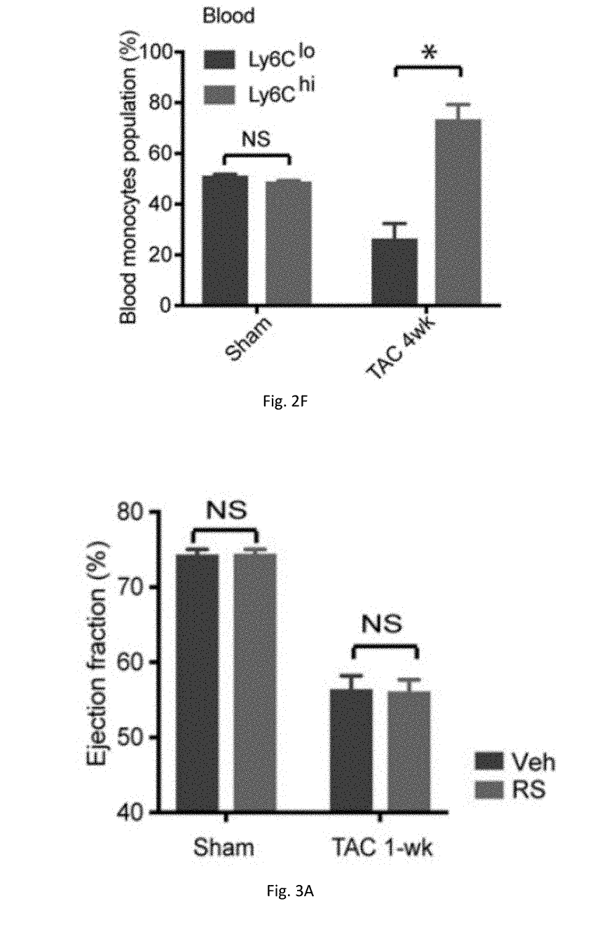

Because TAC increased cardiac macrophage numbers independent of monocyte infiltration at the early stage, we hypothesized that such cells have to be derived from local proliferation. Indeed, we found TAC significantly induced the expression of Ki-67 protein, a nuclear proliferation marker, in cardiac macrophages (FIG. 1I). The local proliferation of cardiac resident macrophages was further confirmed by in vivo BrdU pulse labeling. Mice undergoing sham or TAC surgery were injected i.p. with BrdU for 2 h. Because bone marrow-derived cells are not released into the circulation within this period (FIG. 8), BrdU incorporation in the heart is limited to locally proliferating cells. We observed significant cell proliferation in cardiac macrophages 3 d post-TAC as demonstrated by BrdU incorporation and DNA S-phase populations by flow cytometry (FIGS. 2 A and B). Of note, the pattern of macrophage proliferation (FIG. 2B, blue line) closely aligned with that of cardiac macrophage count (FIG. 2B, red dotted line) along the TAC time line, with proliferation peaking just before the cellular maximum at day 7 and subsequently decreasing to baseline by day 14. Collectively, these studies demonstrate that pressure overload induces local proliferation of cardiac resident macrophages in early-phase POH.

The late-phase monocyte infiltration (FIG. 1H) may result from an expanded circulating monocyte pool and/or greater infiltrative capacity of monocytes. To assess circulating monocyte numbers, we gated blood for monocytes (CD115⁺CD11b⁺) and found that 4-wk TAC (late-phase POH) significantly increased monocyte numbers in blood and spleen but not in bone marrow, indicating TAC-induced extramural monocytosis (FIGS. 2 C and D). Peripheral blood monocytes are classified into three different populations based on expression of cell surface molecules and functions. In humans, these consist of CD14^{hi}CD16− (classic monocytes), CD14^{lo}CD16^{lo} (intermediate monocytes), and CD14^{lo}CD16^{hi} (nonclassic monocytes). In mice, the equivalent populations are Ly6C^{hi}CD62L⁺CD43⁻CCR2⁺ (classic monocytes), Ly6C^{int}CD62L⁻CD43⁺CCR2⁻ (intermediate monocytes), and Ly6C^{lo}CD62L⁻CD43⁺CCR2⁻ (nonclassic monocytes). Given the heterogeneity of these populations, we sought to determine if one population predominantly contributes to monocyte infiltration after TAC. Interestingly, TAC induced an increase in Ly6Chi monocytes in blood but not in spleen or bone marrow (FIGS. 2 E and F). These data demonstrate that pressure overload induces circulating Ly6Chi classic monocyte infiltration during latephase POH.

### Cardiac Resident Macrophages are Required for Myocardial Adaptation in POH

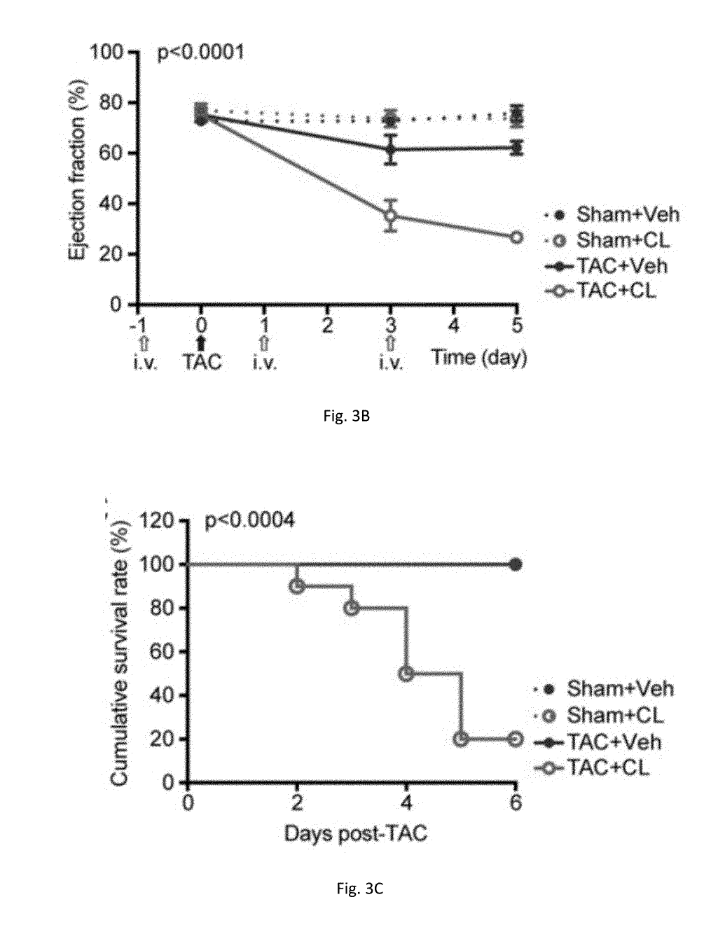

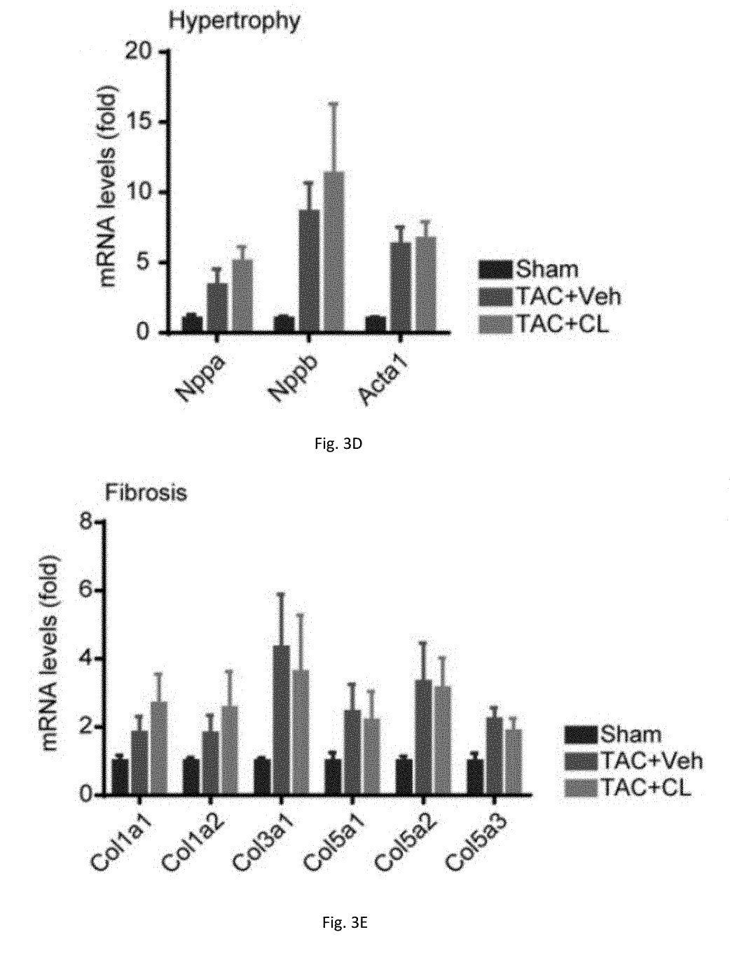

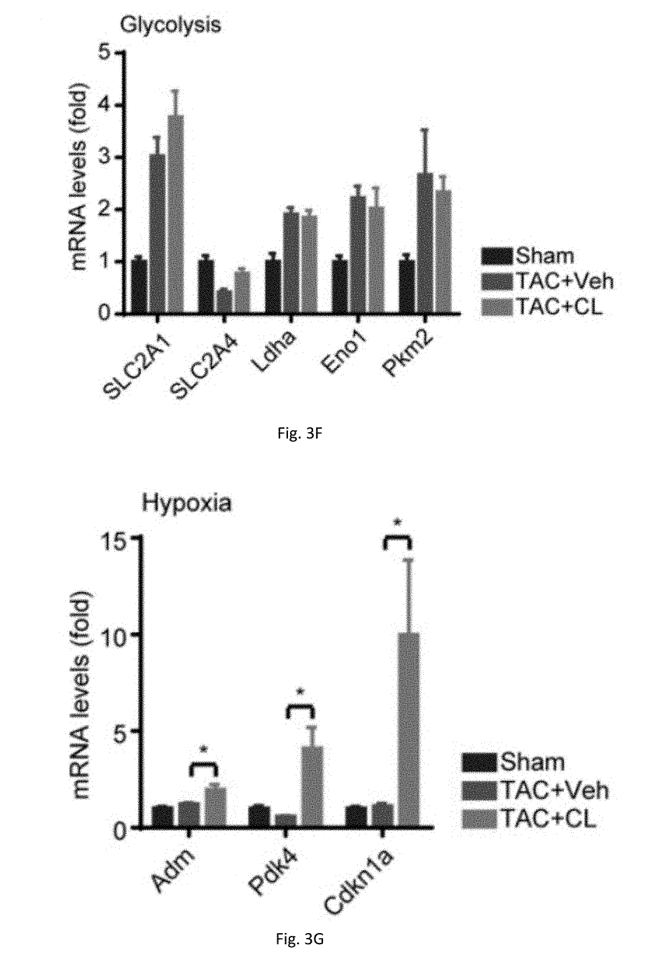

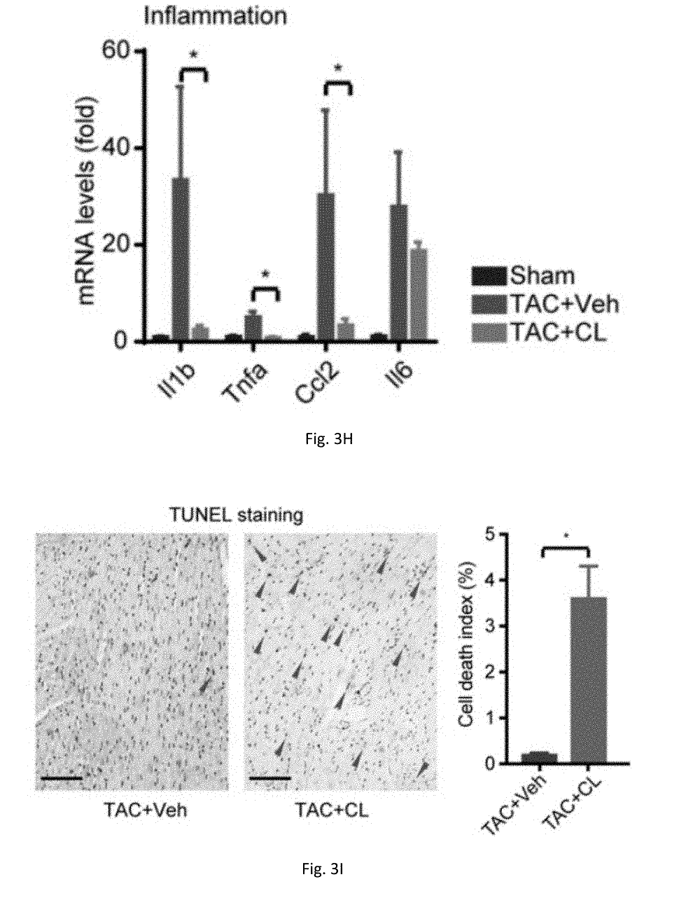

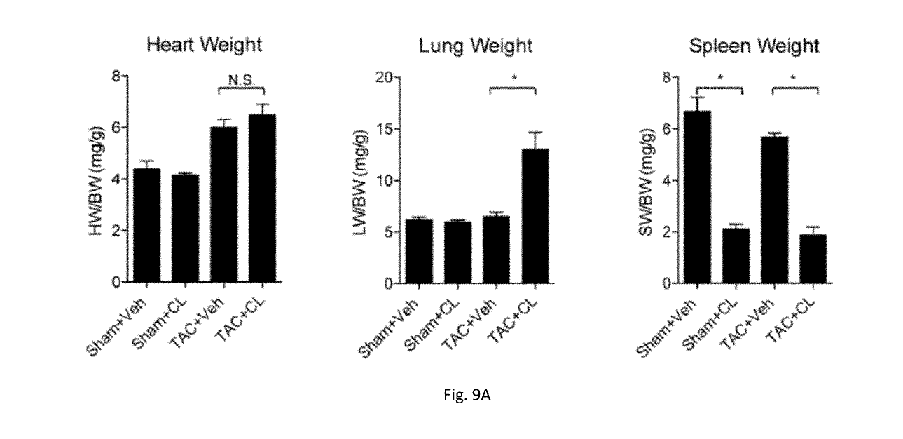

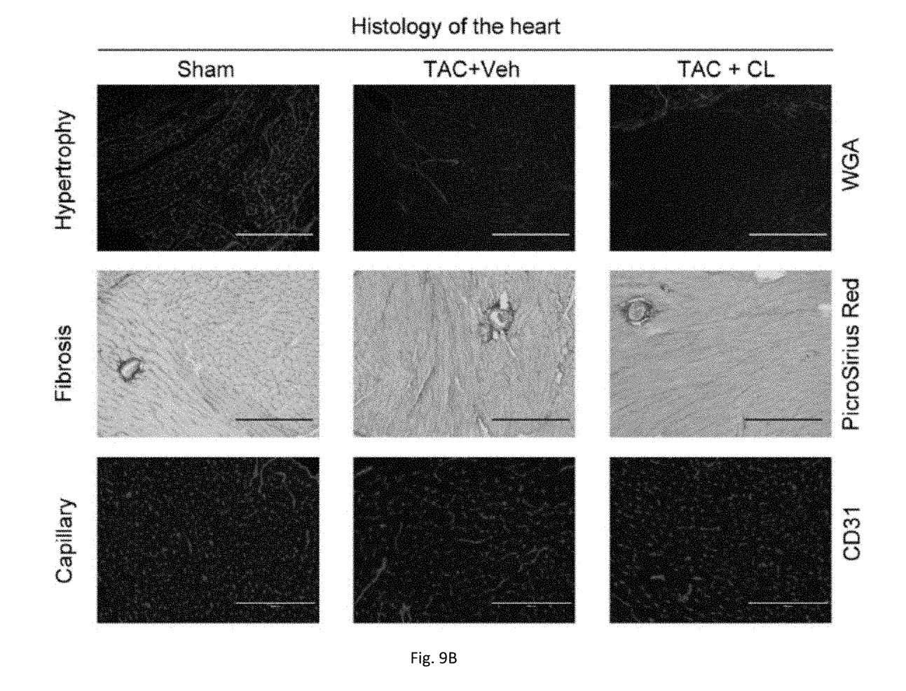

We next sought to determine the functional importance of resident macrophages in the early adaptive response to TAC. As there is no specific method to specifically deplete cardiac resident macrophages, we undertook a stepwise elimination approach to distinguish the unique roles of cardiac resident vs. infiltrating macrophages. We first treated mice with the CCR2 antagonist RS to block infiltration of blood-borne CCR2+Ly6Chi classic monocytes. RS administration had no impact on cardiac macrophage numbers or cardiac function before and after 1 wk post-TAC (FIG. 3A and FIG. 7). Next, we subjected animals to clodronate liposomes (CLs), which deplete both circulating and resident macrophages. Mice with CL administration for a week (500 μg/kg i.v. every other day) did not exhibit any symptom of sickness or heart dysfunction as reported previously. However, when subjected to TAC, the macrophage-depleted animals (CLinjected) developed acute heart failure following TAC as revealed by serial echocardiography studies, while cardiac function was well maintained in PBS-liposome [vehicle (Veh)]-treated animals (FIG. 3B). CL-injected animals also exhibited striking postoperative mortality, with ˜80% dead within 6 d, while a 100% survival rate was observed in all other groups as expected (FIG. 3C). Postmortem autopsy revealed similar heart weight increases after TAC but heavier lungs in the CL-treated TAC group, indicating lung edema secondary to heart failure (FIG. 9A). Spleen weight was similarly reduced by CL in both the sham and TAC groups, confirming efficient macrophage/monocyte depletion (FIG. 9A). Gene expression analysis by quantitative real-time PCR (qPCR) revealed no difference in hypertrophy, fibrosis, or glycolysis genes (FIG. 3 D-F). However, the expression of hypoxia-inducible genes, including Adm, Pdk4, and p21 (Cdkn1a), was significantly higher in the macrophage-depleted TAC (TAC+CL) group compared with the normal TAC (TAC+Veh) group (FIG. 3G), indicating that TAC induced more severe hypoxia stress in the macrophage-depleted myocardium. TAC also induced expression of inflammatory markers, including IL-1β, TNF-α, and CCL2 (MCP1), which was abrogated following macrophage depletion (FIG. 3H), suggesting that inflammation was not the reason for heart failure in early-phase TAC. Histological studies confirmed no significant differences in cardiomyocyte hypertrophy (wheat germ agglutinin staining), fibrosis (collagen staining), or capillary density (CD31 staining) in the myocardium after CL treatment (FIG. 9B). However, we detected profound cardiomyocyte death in macrophage-depleted hearts (FIG. 3I and FIG. 10A). The mechanism of cell death of these cardiomyocytes appeared to be nonapoptotic, as demonstrated by negative caspase-3 activation/cleavage (FIG. 10B). These data underscore the importance of resident macrophages in the early-phase cardiac adaptation to POH.

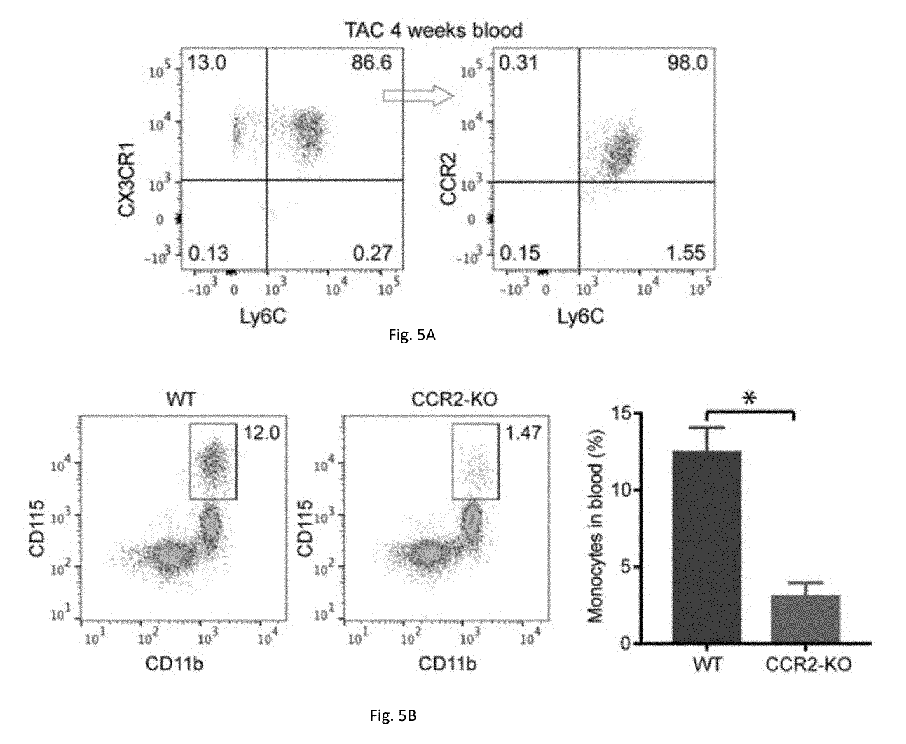

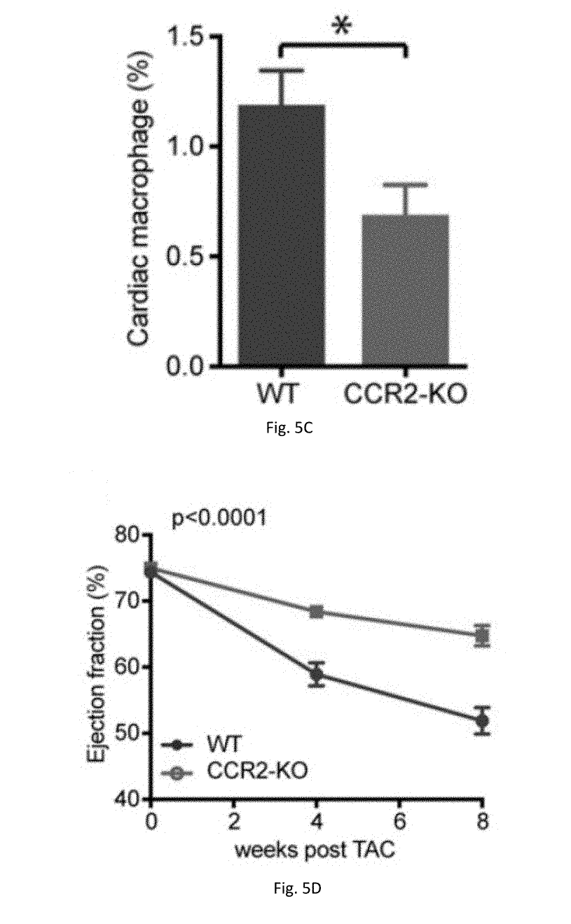

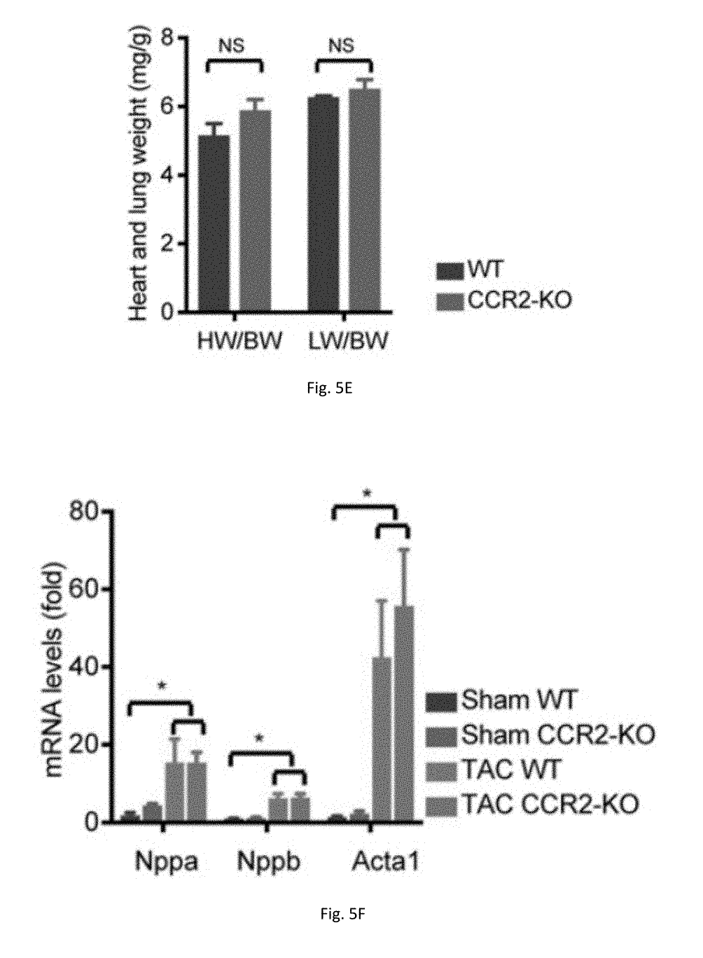

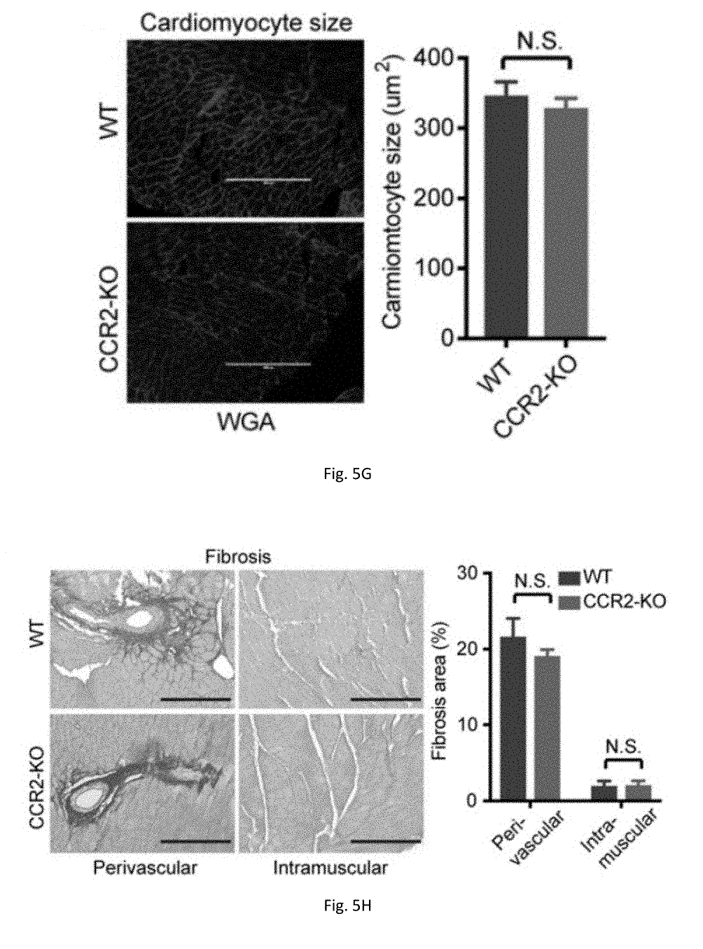

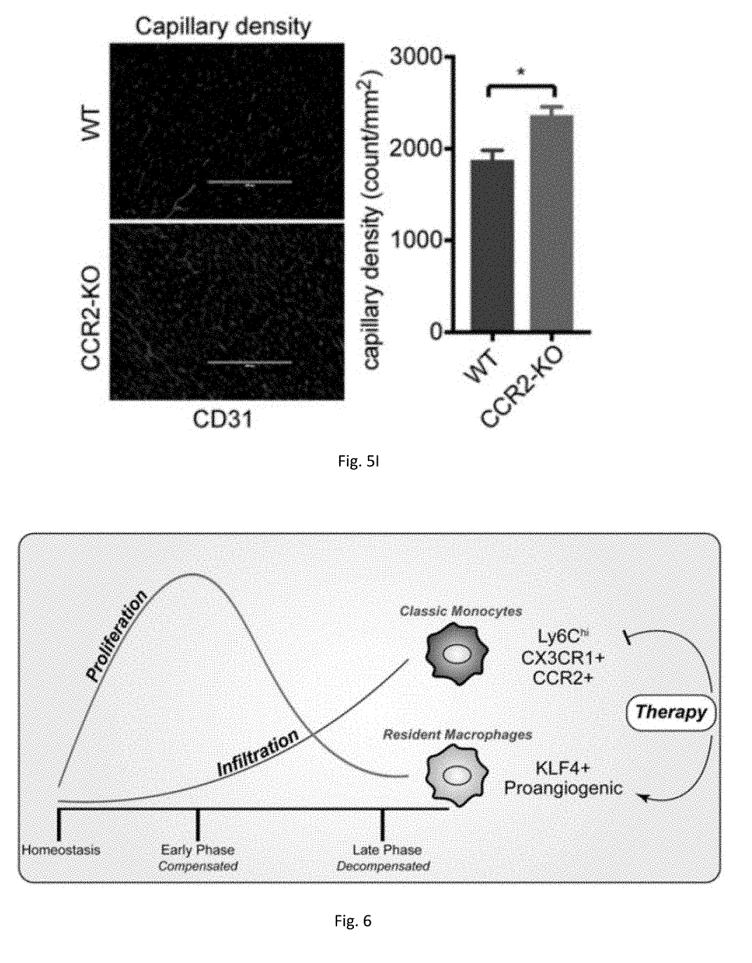

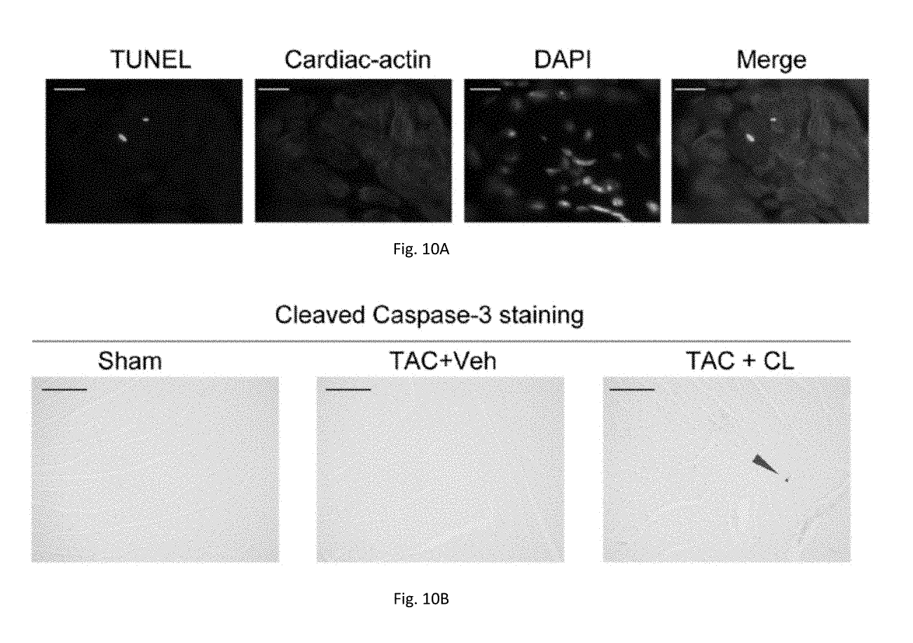

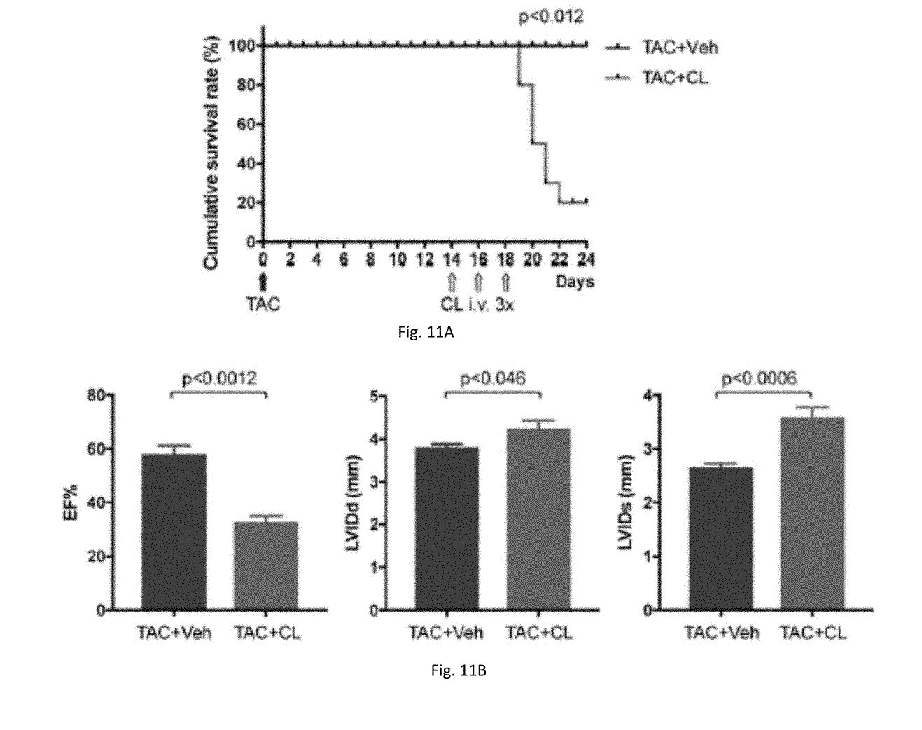

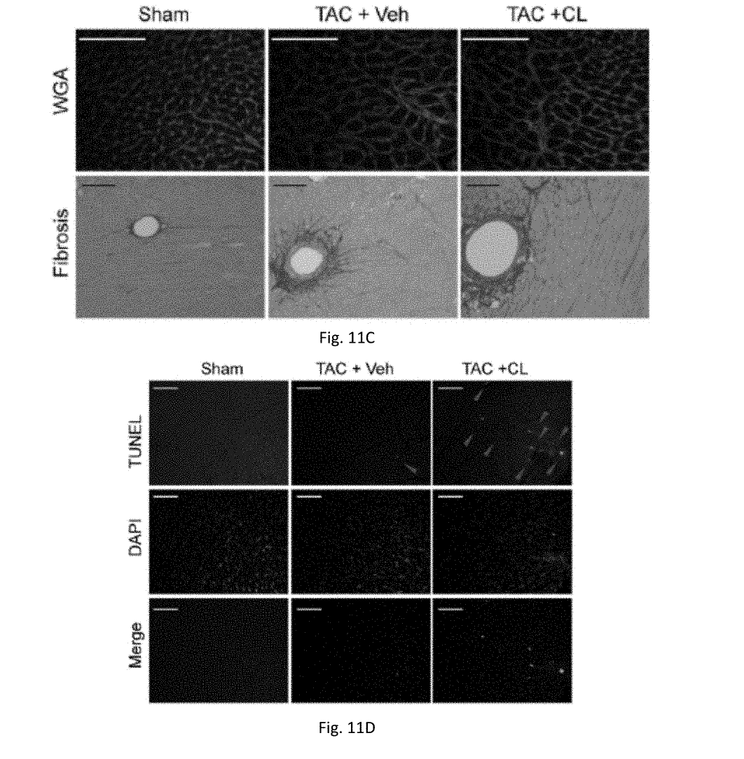

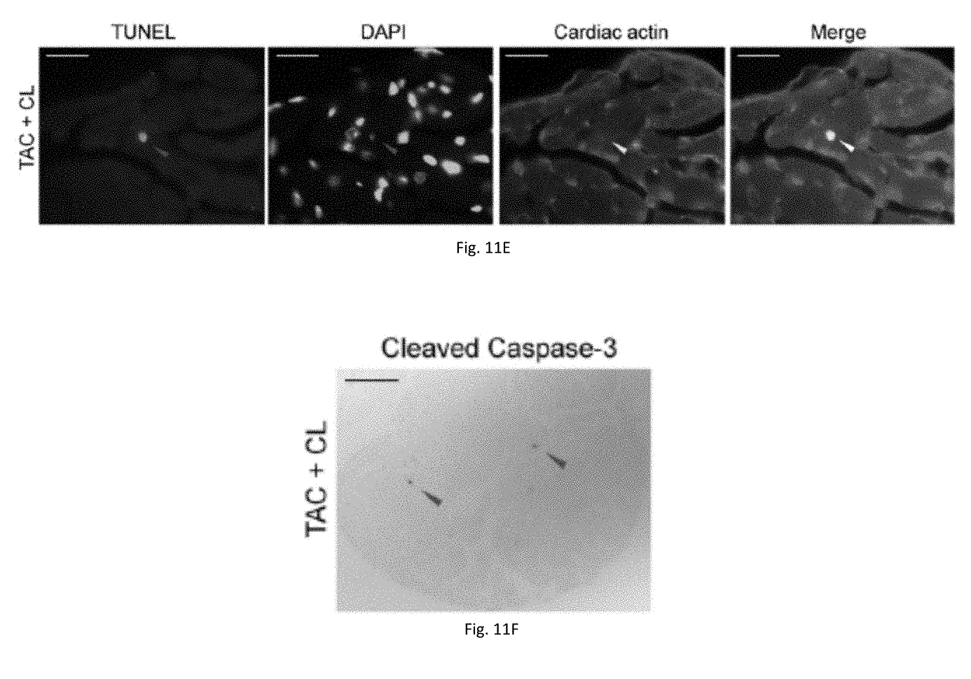

While these data underscore the importance of resident macrophages in early-phase cardiac adaptation to POH, the role of cardiac resident macrophages in late-phase adaptation remains unexplored. To address this, we allowed early compensation to occur after TAC and subsequently depleted macrophages via three alternate-day doses of CL beginning at day 14 post-TAC. As with pre-TAC macrophage depletion, an 80% mortality rate was observed after three injections of CL 3 wk post-TAC (FIG. 11A). It is noteworthy that CL administration trigged a quick transition from cardiac compensation [ejection fraction (EF)˜60%] to heart failure (EF<30%) as revealed by echocardiography (FIG. 11B). CL administration at this point of TAC exhibited little effect on further development of cardiac hypertrophy or fibrosis (FIG. 11C), but resulted in profound cardiomyocyte death (FIG. 11D-F). Because blocking peripheral monocyte infiltration did not promote cardiac dysfunction in early-phase (FIG. 3A) or latephase-TAC (see FIG. 5), these late-stage macrophage depletion studies implicate cardiac resident macrophages in maintaining cardioprotection throughout the POH disease course.

### KLF4 Regulates Proliferation of Cardiac Resident Macrophages

Most tissue resident macrophages, including cardiac resident macrophages, are maintained by self-renewal. While our understanding of how resident macrophages proliferate is limited, a recent seminal study by Sieweke and coworkers provides critical insights. Specifically, it was found that primary macrophages from MafB/c-Maf double-deficient (Maf-DKO) mice demonstrated indefinite KLF4-dependent proliferative capacity when cultured with macrophage colony-stimulating factor (M-CSF). Therefore, we utilized myeloid-specific KLF4-deficient mice (Lyz2-Cre:KLF4^{flox/flox}; designated K4-cKO) to investigate if KLF4 regulates cardiac macrophage proliferation in the context of POH.

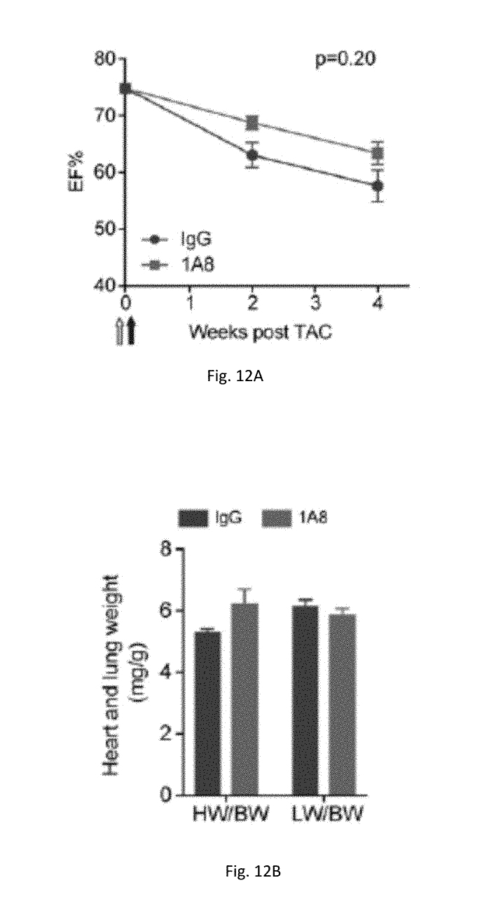

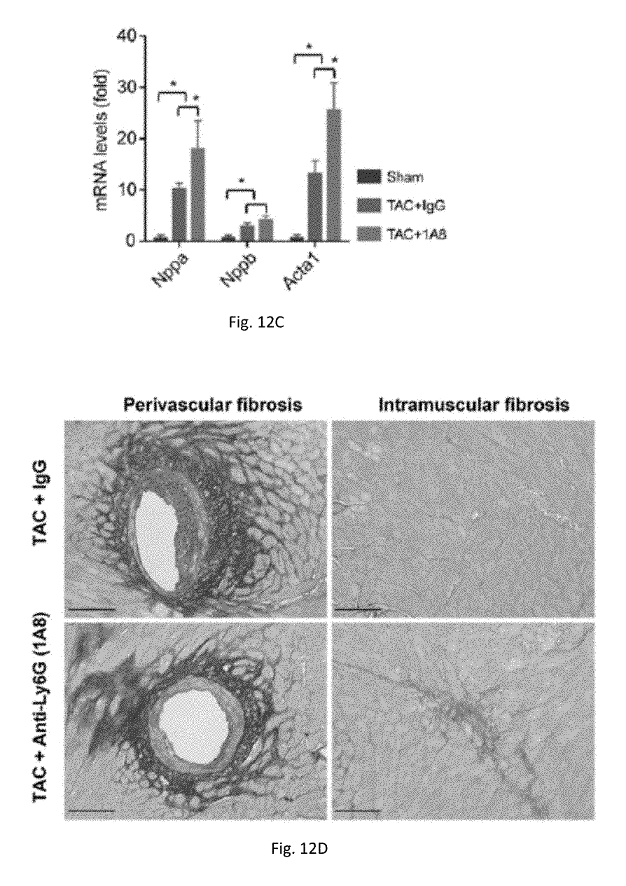

Given that Lyz2-Cre is active in macrophages and neutrophils but not in monocytes, we first sought to explore any potential role for neutrophils in this model. The infiltration of Ly6G+ neutrophils was marginal after TAC (FIG. 1A), and depletion of neutrophils using the anti-Ly6G antibody (clone 1A8) before TAC demonstrated very little effect on POH (FIG. 12). Therefore, these data supported our decision to focus on the role of KLF4-deficient macrophages.

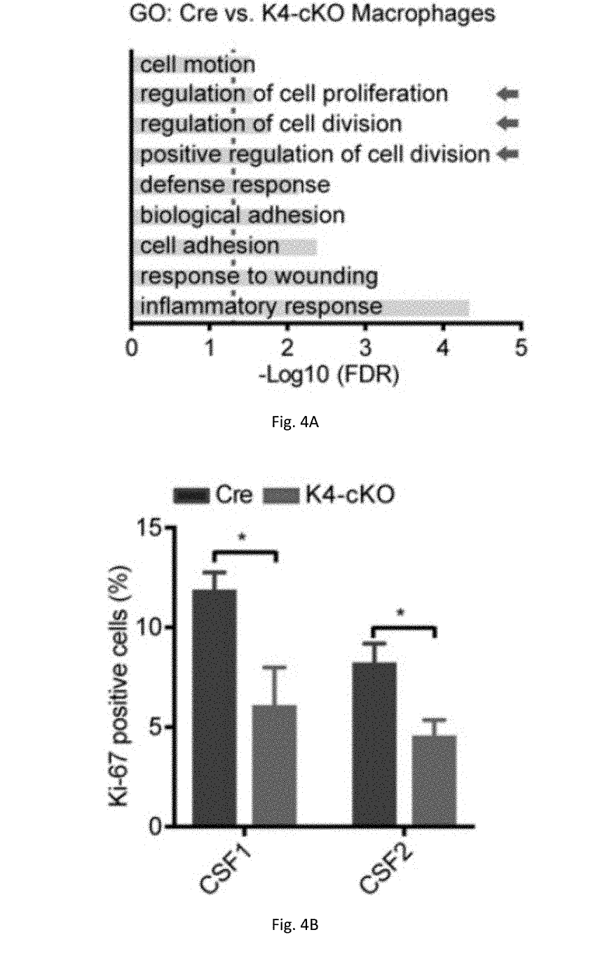

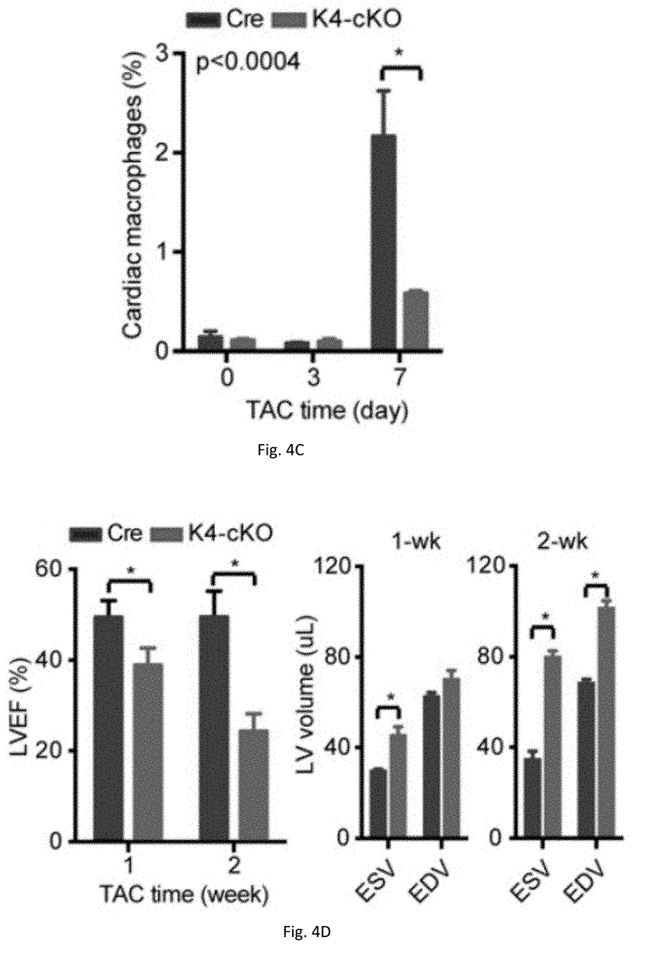

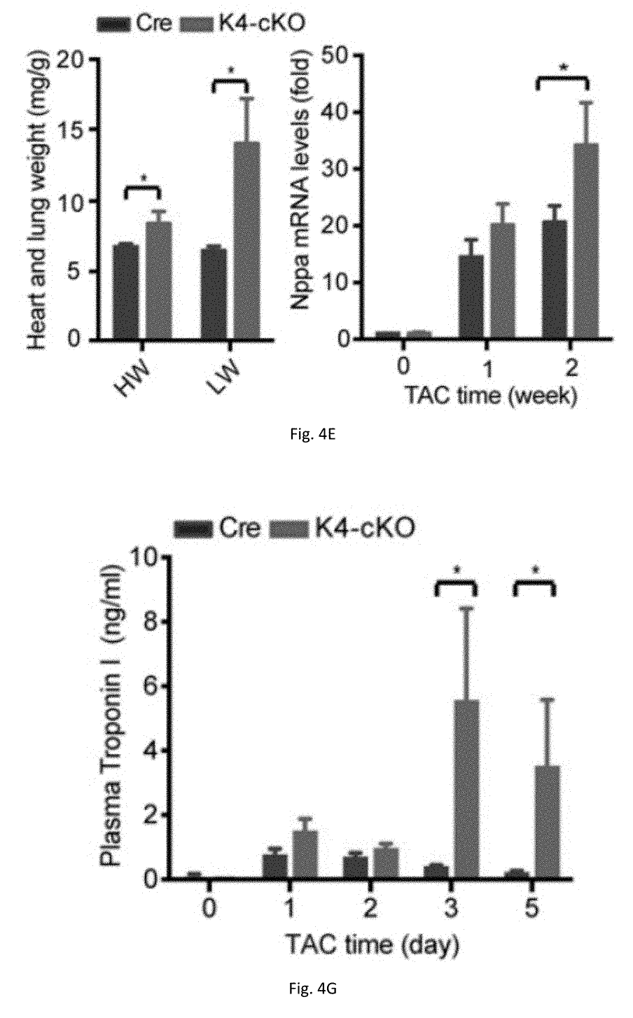

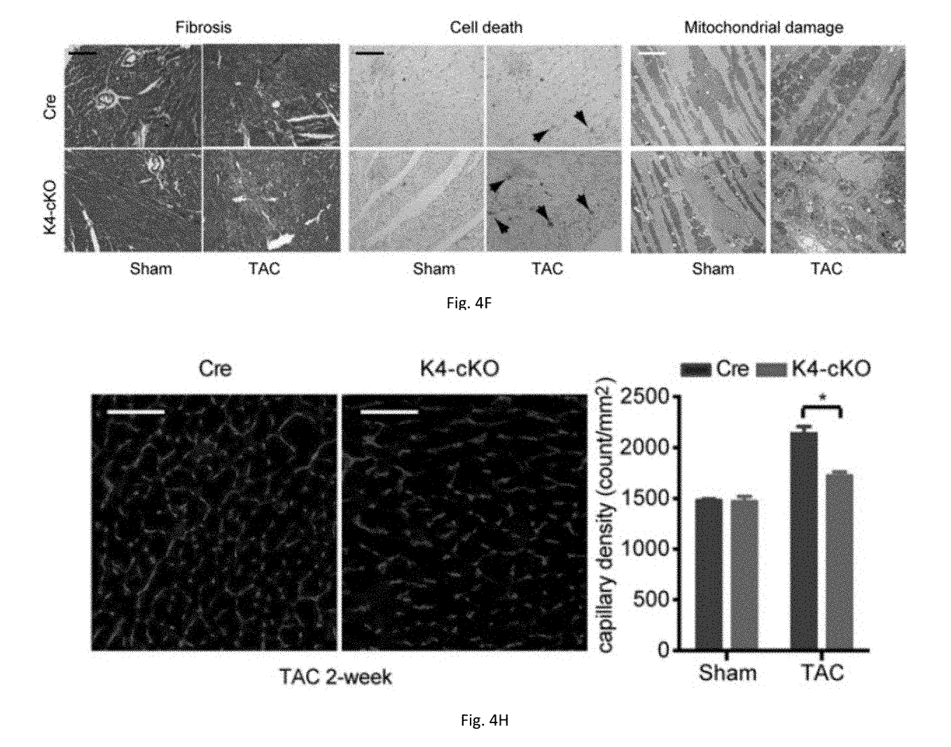

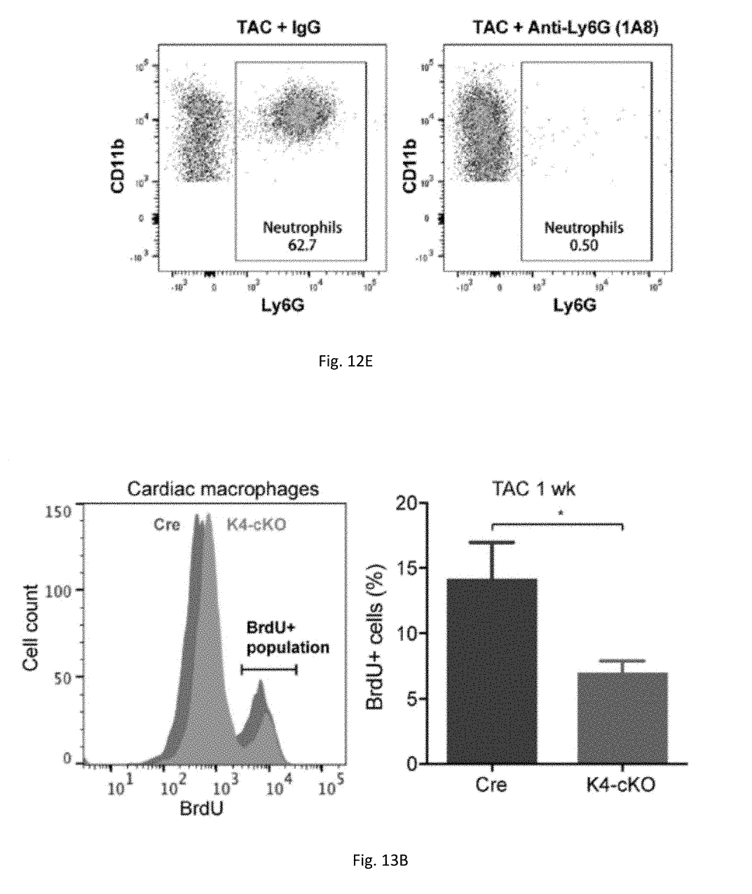

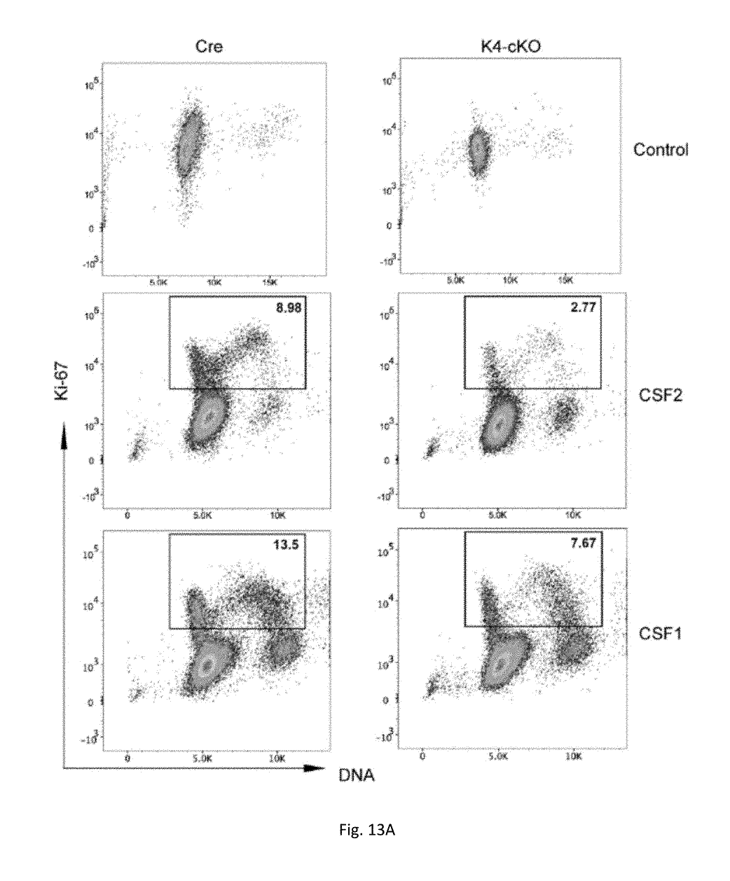

We first performed transcriptomic analysis of primary macrophages from Lyz2-Cre (designated Cre) and K4-cKO mice via RNA-sequencing (RNA-seq) and found 238 genes that were differentially expressed (false discovery rate<0.05). Gene ontology (GO) analysis demonstrated enrichment in multiple pathways of cell proliferation and division (FIG. 4A). Importantly, when treated in vitro with CSF1 and CSF2, KLF4-deficient primary macrophages showed less accumulation of Ki-67 protein (FIG. 4B and FIG. 13A), demonstrating impaired proliferative capacity. Next, we subjected the K4-cKO and Cre animals to TAC. FACS analysis demonstrated that TAC-induced cardiac macrophage accumulation was significantly reduced in the K4-cKO group compared with that of Cre (FIG. 4C). Consistent with fewer cardiac macrophages, myeloid KLF4 deficiency impaired cardiac resident macrophage proliferation in vivo following TAC as demonstrated by reduced BrdU incorporation (FIG. 13B).

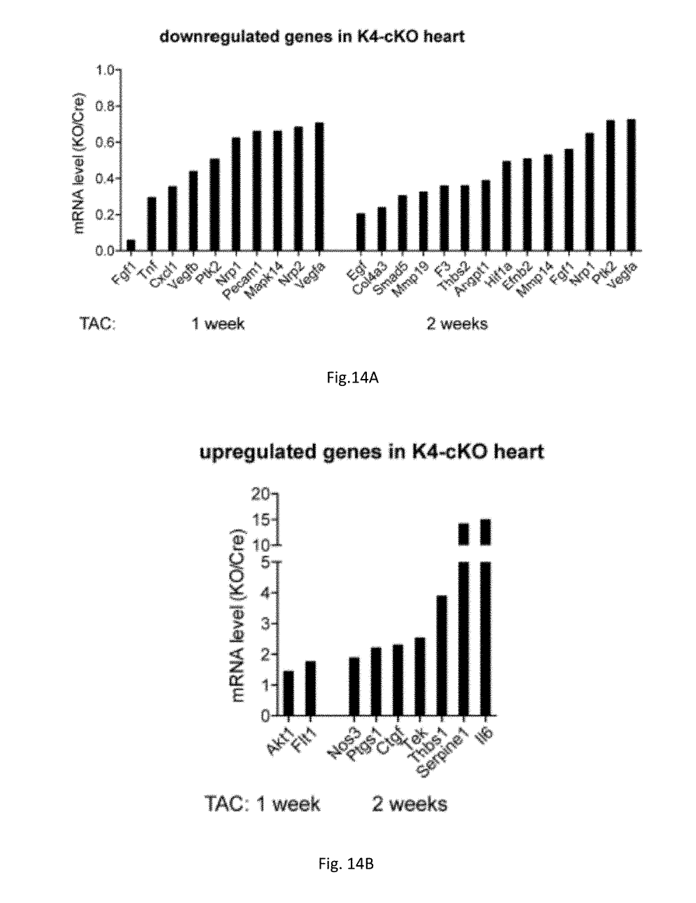

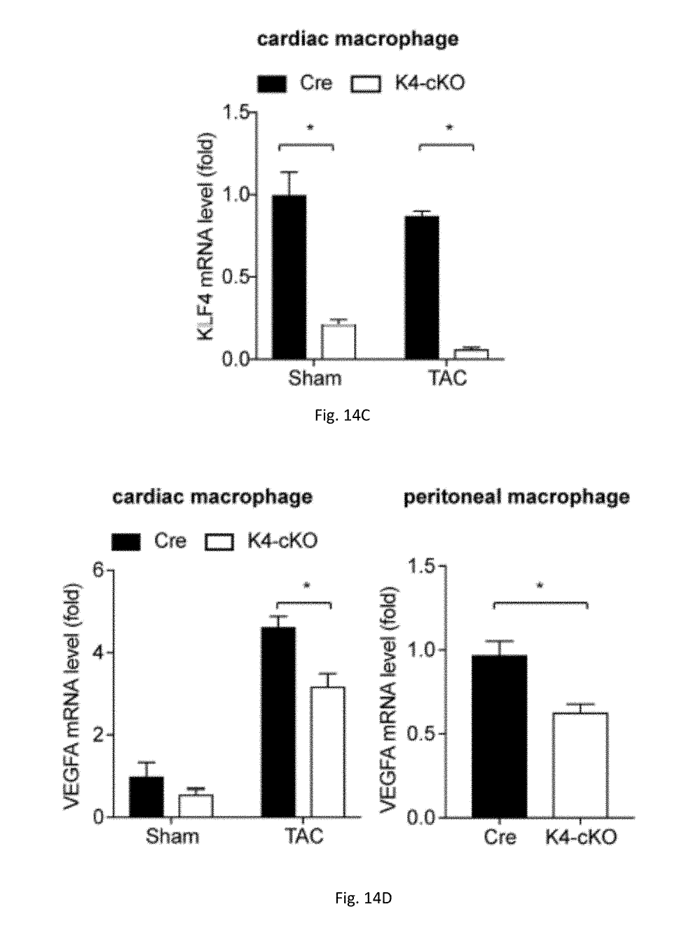

Post-TAC serial echocardiography revealed that K4-cKO mice rapidly develop a dilated cardiomyopathy characterized by reduced left ventricular EF (FIG. 4D), elevated heart and lung weight, and expression of the cardiac hypertrophy marker gene Nppa (FIG. 4E). Histological analyses exhibited enhanced fibrosis and cell death, as well as severe mitochondrial damage in K4-cKO hearts post-TAC (FIG. 4F). Plasma cardiac troponin I levels were significantly higher in K4-cKO mice post-TAC, indicating myocardial injury (FIG. 4G). K4-cKO hearts exhibited significantly impaired angiogenesis as demonstrated by CD31 immunostaining and the dysregulation of angiogenesis genes (FIG. 4H and FIGS. 14A and B). Although not induced by TAC, macrophage KLF4 appeared to be required for optimal expression of a potent angiogenesis factor, VEGFA, in vitro and in the heart (FIGS. 14C and D). These data, paired with the observation that many cardiac macrophages reside in the perivascular region (FIG. 1C), suggest that cardiac macrophages may have a potential role in angiogenesis or other vascular responses. In addition to impaired angiogenesis, we observed that K4-cKO hearts exhibited enhanced fibrosis after TAC (FIG. 4F). However, qPCR analysis using a panel of 84 fibrosis-related genes (Qiagen RT2 Profiler PCR array) revealed no significant difference between Cre and K4-cKO cardiac macrophages (FIG. 14E). These observations, coupled with the appreciation of reduced KLF4-deficient cardiac macrophage numbers (FIG. 4C), suggest that enhanced fibrosis seen in the K4-cKO heart was likely secondary to myocardial injury (FIGS. 4 F and G).

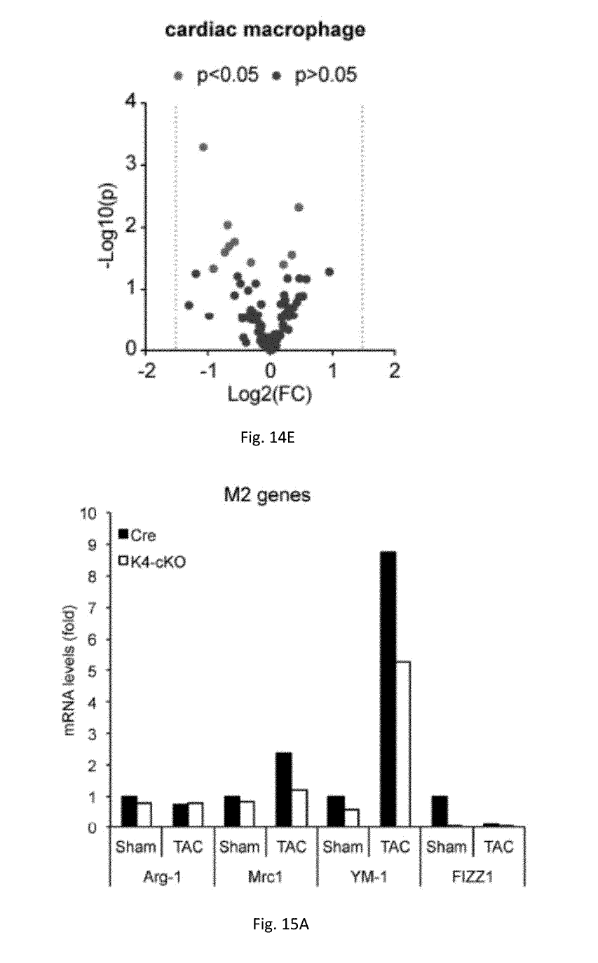

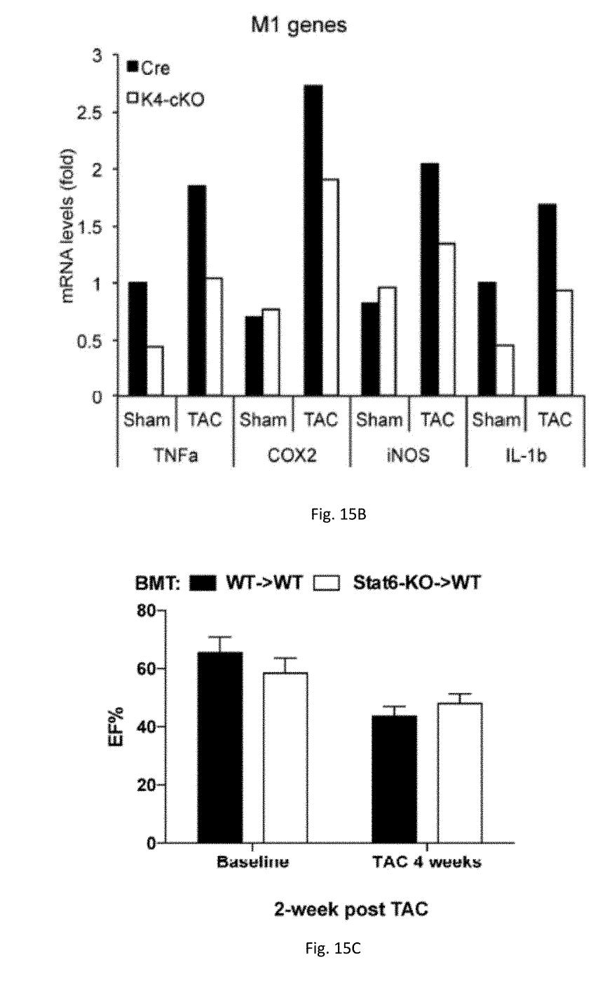

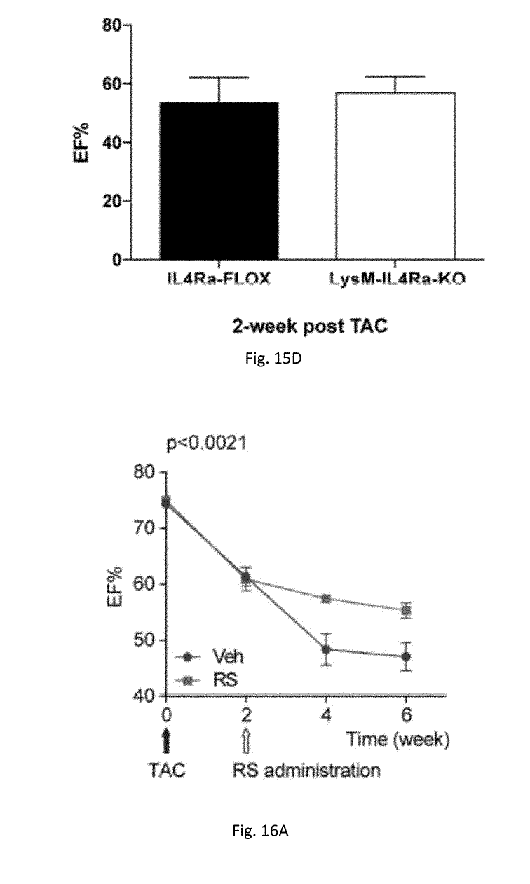

Because KLF4 also regulates macrophage M1/M2 polarization and gene expression in KLF4-deficient macrophages exhibited enriched inflammation pathways (FIG. 4A), we tested if the macrophage polarization state contributes to the phenotype observed after TAC. Consistent with our previous report (24), K4-cKO hearts at 1 wk post-TAC showed lower expression of multiple M2 genes (FIG. 15A). However, the expression of M1 genes in K4-cKO hearts was also lower, indicating the absence of excessive inflammation (FIG. 15B). We attribute this phenomenon to reduced cardiac macrophage numbers in K4-cKO hearts after TAC (FIG. 4C). We further studied two classic M2-deficient animals, namely, hematopoietic STAT6-KO mice (via transplantation of STAT6-KO bone marrow to WT recipients) and myeloid IL4Ra-KO mice (Lyz2-Cre:IL4Raflox/flox), and found that biased M1/M2 polarization had no effect on POH (FIGS. 15C and D). As such, we conclude that the role of KLF4 in cardiac macrophages in POH is not simply defined by M1/M2 polarization but is secondary to its regulation of cardiac resident macrophage proliferation and their angiogenic potential (FIG. 4C and FIG. 14D).

### Infiltrating Macrophages are Detrimental to the Heart in Late-Phase POH

While our studies indicate that blood-borne macrophages are not required for the early-phase protective compensatory response to POH, they do infiltrate the myocardium during latestage POH (FIG. 1G). Because heart failure is often associated with immune cell infiltration and inflammation, we asked if blood-borne macrophages contribute to late-phase decompensation of POH. As shown in FIG. 2F, TAC increased circulating Ly6Chi monocytes during late-phase POH. These monocytes were further characterized as Ly6C^{hi}CX3CR1⁺CCR2⁺ classic monocytes (FIG. 5A). To block such monocytes, we utilized the CCR2-KO mouse, which is known to have reduced circulating classic monocytes due to impaired CCL2/CCR2-mediated trafficking from the bone marrow. When subjected to 4-wk TAC, the circulating monocytes remained much lower (˜25%) in CCR2-KO mice than WT control mice (FIG. 5B). Consistently, overall macrophage numbers in latephase POH were also lower (˜50%) within CCR2-KO myocardium (FIG. 5C).

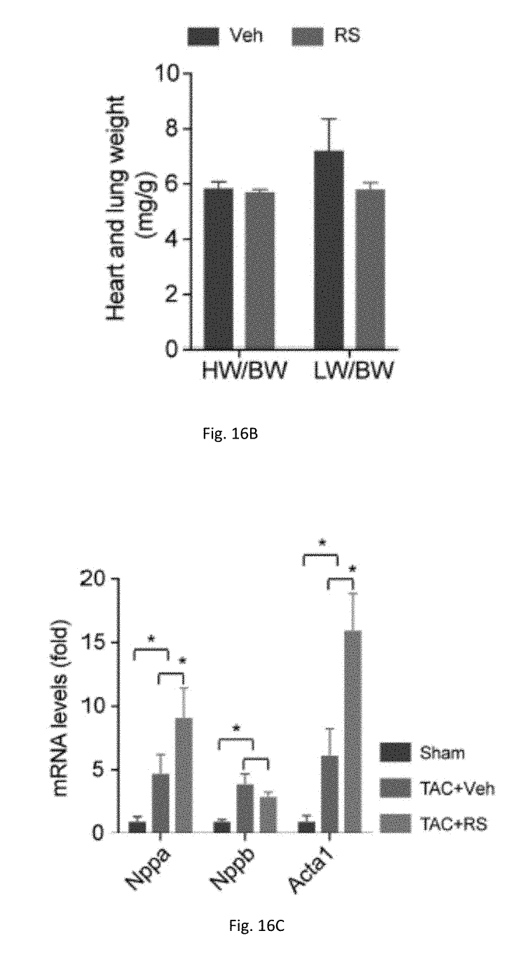

Serial echocardiography for 8 wk post-TAC demonstrated that WT mice gradually develop cardiac dysfunction during latephase POH, while CCR2-KO mice maintain relatively normal cardiac function (EF at 8 wk post-TAC: WT: 52±2.0% vs. CCR2-KO: 65±1.5%; P<0.0001) (FIG. 5D). Although there was no difference in cardiac hypertrophy or fibrosis between WT and CCR2-KO groups (FIG. 5 E-H), myocardial capillary density was significantly higher in the CCR2-KO group (capillary counts per 1 mm2 of myocardium: WT: 1,872±113 vs. CCR2-KO: 2,359±98; ˜26% increase; P<0.0038) (FIG. 5I), suggesting that CCR2-KO mice were able to maintain superior angiogenesis during the 8 wk of TAC. Finally, blocking CCR2 signaling in WT animals using RS preserved cardiac function in an established POH model (RS oral administration 2 wk after TAC) without affecting cardiac hypertrophy (FIG. 16). Collectively, these data demonstrate that blocking monocyte/macrophage infiltration maintains angiogenesis and preserves cardiac function without affecting cardiac hypertrophy or fibrosis in latephase POH. Taken together, these data demonstrate that latephase infiltration of Ly6ChiCX3CR1+CCR2+ classic monocytes contributes to cardiac dysfunction in POH and, conversely, that blockade of infiltrating macrophages (genetically by CCR2-KO or pharmaceutically by CCR2 antagonist) preserves heart function, in part, through improved angiogenesis.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, we claim:

1. A method of treating a cardiomyopathy in a subject in need thereof, the method comprising:
administering to the subject a therapeutically amount of a CCR2 inhibitor.

2. The method of claim 1, wherein the CCR2 inhibitor is administered at an amount effective to preserve cardiac function.

3. The method of claim 1, wherein the cardiomyopathy is non-ischemic cardiomyopathy.

4. The method of claim 1, wherein the CCR2 inhibitor is administered to the subject after the occurrence of the cardiomyopathy to inhibit infiltrations of blood-borne macrophages into myocardium of the subject.

5. The method of claim 1, wherein the blood-borne macrophages include Ky6C^{hi}, CX3CR1⁺, CCR2⁺ macrophages.

6. The method of claim 1, wherein the CCR2 inhibitor is selected from the group consisting of: (i) a direct CCR2 antagonist; (ii) an inverse CCR2 agonist; (iii) a negative allosteric CCR2 modulator; (iv) an indirect CCR2 antagonist; (v) an indirect inverse CCR2 agonist; and (vi) an indirect negative allosteric CCR2 modulator.

7. The method of claim 1, wherein the CCR2 inhibitor is selected from the group consisting of RS504393, RS102895, MLN-1202, INCB8696, MK-0812, CCX140, PF-4136309, and BMS-741672.

8. The method of claim 1, wherein the CCR2 inhibitor is administered to the subject during late-phase pressure overload hypertrophy.

9. The method of claim 1, further comprising administering an agent to the subject that promotes expression Kruppel-like factor 4 (KLF4) in myocardium of the subject.

10. The method of claim 8, wherein the agent is administered to the subject at an amount effective to cardiac resident macrophage proliferation and angiogenic activities.

11. The method of claim 8, wherein the agent comprises a vector encoding KLF4.

12. A method of treating a nonischemic cardiomyopathy in a subject in need thereof, the method comprising:
administering to the subject a therapeutically amount of a CCR2 inhibitor.

13. The method of claim 12, wherein the CCR2 inhibitor is administered at an amount effective to preserve cardiac function without affecting cardiac hypertrophy.

14. The method of claim 12, wherein the CCR2 inhibitor is administered to the subject after the occurrence of the cardiomyopathy to inhibit infiltrations of blood-borne macrophages into myocardium of the subject.

15. The method of claim 12, wherein the CCR2 inhibitor is selected from the group consisting of: (i) a direct CCR2 antagonist; (ii) an inverse CCR2 agonist; (iii) a negative allosteric CCR2 modulator; (iv) an indirect CCR2 antagonist; (v) an indirect inverse CCR2 agonist; and (vi) an indirect negative allosteric CCR2 modulator.

16. The method of claim 12, wherein the CCR2 inhibitor is selected from the group consisting of RS504393, RS102895, MLN-1202, INCB8696, MK-0812, CCX140, PF-4136309, and BMS-741672.

17. The method of claim 12, wherein the CCR2 inhibitor is administered to the subject during late-phase pressure overload hypertrophy.

18. The method of claim 12, further comprising administering an agent to the subject that promotes expression Kruppel-like factor 4 (KLF4) in myocardium of the subject.

19. The method of claim 18, wherein the agent is administered to the subject at an amount effective to cardiac resident macrophage proliferation and angiogenic activities.

20. The method of claim 18, wherein the agent comprises a vector encoding KLF4.
